(19) United States
(12) Patent Application Publication
(10) Pub. No.: US 2010/0298530 A1
(43) Pub. Date: Nov. 25, 2010

(54) Photoresponsive Base Having Triazole Skeleton

(75) Inventors: KENZOU FUJIMOTO, ISHIKAWA (JP); TAKEHIRO AMI, ISHIKAWA (JP); CHIFUMI NAGATA, ISHIKAWA (JP); AKIO YAMANE, TOKYO (JP)

Correspondence Address: SQUIRE, SANDERS & DEMPSEY L.L.P., 275 BATTERY STREET, SUITE 2600, SAN FRANCISCO, CA, 94111-3356, US

(73) Assignee: TOPPAN PRINTING CO., LTD., TOKYO (JP)

(21) Appl. No.: 12/780,735

(22) Filed: May 14, 2010

Related U.S. Application Data
(63) Continuation of application No. PCT/JP2008/070884, filed on Nov. 17, 2008.

(30) Foreign Application Priority Data
Nov. 19, 2007 (JP) 2007-299901

(51) Int. Cl. C07K 9/00; C07D 249/04

(52) U.S. Cl. 530/322; 548/255

(57) ABSTRACT

To provide a photocrosslinkable compound and a photocrosslinking agent which are capable of ligation of nucleic acids within a short period of time compared to the conventional cases, and to which modifications depending on the intended use can easily be made, and a method of producing the photocrosslinking agent. Nucleic acids including a group represented by the formulae I, III, IV, or V as a nucleobase; a photocrosslinking agent including the nucleic acids, a method of producing nucleic acids by reacting nucleic acids including a group represented by the formulae VI, VII, VIII, or IX as a nucleobase with an aromatic azidated product represented by the formula X.

### CROSS REFERENCE TO RELATED APPLICATION

This application is a continuation of PCT International Application No. PCT/JP2008/070884, the entire disclosure of which is incorporated herein by reference.

### TECHNICAL FIELD

The present invention relates to a photoresponsive base having a triazole skeleton, and also relates to nucleic acids including the photoresponsive base and the production method thereof.

### BACKGROUND ART

The crosslinking of nucleic acids is one of the basic techniques in the field of molecular biology. For example, the crosslinking of nucleic acids is used for introducing a gene or detecting a base sequence by being combined with a hybridization technique. For this reason, the crosslinking of nucleic acids is an extremely important technique which is used not only in the basic researches of molecular biology, but also, for example, in the diagnosis and therapeutics in the medical field, the development and production of therapeutic agents, diagnostic reagents or the like, and the development and production of enzymes, microorganisms or the like in the industrial and agricultural fields.

The crosslinking of nucleic acids has conventionally been carried out, for example, by the use of a DNA ligase or the like. However, when adopting such an enzymatic reaction mimicking an in vivo reaction, a special setting is required for the reaction conditions, and there are also some further disadvantages regarding the enzymes used, such as their relatively high cost and their poor stability. In order to overcome such disadvantages, intensive and extensive studies have been conducted on the techniques for ligation of nucleic acids without the use of enzymes.

As a technique for ligation of nucleic acids without using enzymes, there is a method using an organic compound which is reactive with nucleic acids. In recent years, nucleic acid crosslinking techniques involving a photoreaction has been attracting attention in view of the advantages, for example, the reaction can be freely controlled both temporally and spatially, and the reaction can be conducted under more relaxed conditions compared to those of general organic chemical reactions.

As such a nucleic acid crosslinking technique, a photocrosslinking technique using 5-cyanovinyldeoxyuridine has been known (Patent Document 1: Japanese Patent No. 3,753,938, Patent Document 2: Japanese Patent No. 3,753,942).

Patent Document 1: Japanese Patent No. 3,753,938

Patent Document 2: Japanese Patent No. 3,753,942

### SUMMARY OF THE INVENTION

However, in the conventional photocrosslinking technique described above, the crosslinking required a reaction time of a few minutes to a few tens of minutes, and thus the technique was not superior to the crosslinking of nucleic acids through the enzymatic reaction from this viewpoint.

Accordingly, an object of the present invention is to provide a method for ligation of nucleic acids without the use of an enzyme and under relaxed conditions within a short period of time compared to the conventional cases, and a compound and a crosslinking agent which can be used for the method.

In addition, another object of the present invention is to provide a production method of a crosslinking agent which can be used for the above-mentioned method, and a compound and a modifying agent which can be used for the production method.

Further, it was necessary to subject the photocrosslinkable compounds used in the conventional photocrosslinking technique to a reaction in advance, under reaction conditions commonly adopted in organic synthesis, in order to carry out chemical modifications for providing the compounds with photocrosslinking properties as well as the labeled sites for detection due to the chemical structures thereof. However, the chemical modifications for providing the compounds with photocrosslinking properties as well as the labeled sites for detection were not easy procedures, and thus the application thereof as photocrosslinking agents were restricted as a result.

Therefore, it is still another object of the present invention to provide a photocrosslinkable compound and a photocrosslinking agent which can be easily obtained, compared to conventional photocrosslinking agents, by carrying out chemical modifications for provision of photocrosslinking properties, labeled sites for detection, and the like, and which include structures capable of easily obtaining derivatives in which the modifications depending on the intended use have been made.

In addition, it is yet another object of the present invention to provide a production method of the above-mentioned photocrosslinking agent, and a compound and a modifying agent which can be used for the production method.

The present inventors and others have conducted intensive and extensive studies on the photocrosslinking agent for ligation of nucleic acids and discovered that the use of a photocrosslinking agent including nucleic acids using a photoresponsive base having a triazole skeleton structure according to the present invention can achieve the above-mentioned objects.

Accordingly, the present invention includes the following aspects [1] to [4].

[1] Nucleic acids which include a nucleic acid as well as a peptide nucleic acid, the nucleic acids including a group represented by the following formulae I, III, IV, or V as a nucleobase:

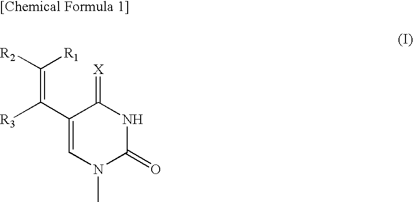

(with the proviso that in the formula I, X represents O, S or NH;

each of R₁ and R₃ independently represents hydrogen, an alkyl group of 1 to 6 carbon atoms, an alkoxy group of 1 to 6 carbon atoms, a cyano group, or an acyl group of 1 to 6 carbon atoms; and

R₂ is a group represented by the following formula II:

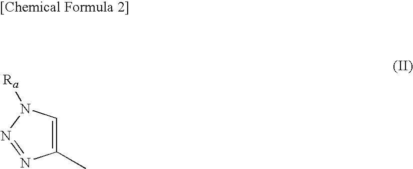

(with the proviso that in the formula II, Ra represents a substituent which can be conjugated with a triazole structure));

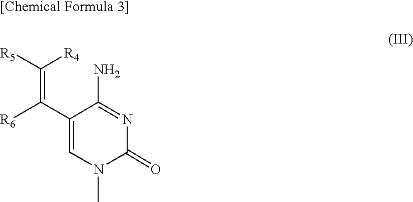

(with the proviso that in the formula III, each of R₄ and R₆ independently represents hydrogen, an alkyl group of 1 to 6 carbon atoms, an alkoxy group of 1 to 6 carbon atoms, a cyano group, or an acyl group of 1 to 6 carbon atoms;

and R₅ is a group represented by the formula II (with the proviso that in the formula II, Ra represents a substituent which can be conjugated with a triazole structure));

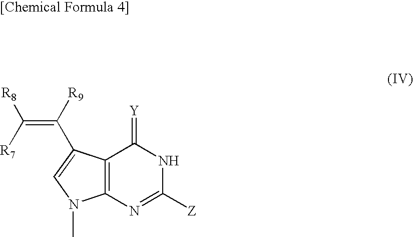

(with the proviso that in the formula IV, Y represents O, S or NH;

Z represents NH₂ when Y represents O or S, and represents a hydrogen atom when Y represents NH;

each of R₇ and R₉ independently represents hydrogen, an alkyl group of 1 to 6 carbon atoms, an alkoxy group of 1 to 6 carbon atoms, a cyano group, or an acyl group of 1 to 6 carbon atoms;

and R₈ is a group represented by the following formula II (with the proviso that in the formula II, Ra represents a substituent which can be conjugated with a triazole structure));

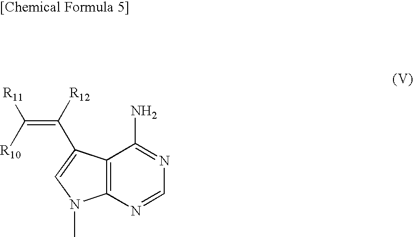

(with the proviso that in the formula V, each of R₁₀ and R₁₂ independently represents hydrogen, an alkyl group of 1 to 6 carbon atoms, an alkoxy group of 1 to 6 carbon atoms, a cyano group, or an acyl group of 1 to 6 carbon atoms;

and R₁₁ is a group represented by the following formula II (with the proviso that in the formula II, Ra represents a substituent which can be conjugated with a triazole structure)).

[2] The nucleic acids according to claim 1, wherein Ra is a monovalent group of an aromatic group, having 1, 2, 3 or more rings.

[3] A photocrosslinking agent including the nucleic acids according to the aspect [1].

[4] A method for photocrosslinking of nucleic acids using the nucleic acids according to the aspect [1].

In the preferred embodiment, Ra represents a monovalent group of a substituted or unsubstituted aromatic compound, and includes several rings typically within a range from 1 to 10, preferably from 1 to 8, more preferably from 1 to 6, still more preferably from 1 to 4, and particularly preferably from 1 to 3. In addition, the aromatic compound may be a heterocyclic compound.

In the preferred embodiment, Ra represents a monovalent group of a substituted or unsubstituted aromatic compound, and a monovalent group of benzene, pentalene, indene, naphthalene, azulene, heptalene, biphenylene, as-indacene, s-indacene, acenaphthylene, fluorene, phenalene, phenanthrene or anthracene.

In the preferred embodiment, Ra is formed typically from a 4 to 8-membered ring, preferably from a 4 to 7-membered ring, more preferably from a 4 to 6-membered ring, still more preferably from a 5 to 6-membered ring, and particularly preferably from a 6-membered ring.

In the preferred embodiment, Ra represents a group selected from the group consisting of benzen-1-yl (phenyl group), pentalen-1-yl, pentalen-2-yl, pentalen-3-yl, inden-2-yl, inden-3-yl, inden-4-yl, inden-5-yl, inden-6-yl, inden-7-yl, naphthalen-1-yl, naphthalen-2-yl, azulen-1-yl, azulen-2-yl, azulen-3-yl, azulen-4-yl, azulen-5-yl, azulen-6-yl, azulen-7-yl, azulen-8-yl, heptalen-1-yl, heptalen-2-yl, heptalen-3-yl, biphenylen-1-yl, biphenylen-2-yl, as-indacen-1-yl, as-indacen-2-yl, as-indacen-3-yl, as-indacen-4-yl, as-indacen-5-yl, as-indacen-6-yl, as-indacen-7-yl, as-indacen-8-yl, s-indacen-1-yl, s-indacen-2-yl, s-indacen-3-yl, s-indacen-4-yl, acenaphthylen-1-yl, acenaphthylen-3-yl, acenaphthylen-4-yl, acenaphthylen-5-yl, fluoren-1-yl, fluoren-2-yl, fluoren-3-yl, fluoren-4-yl, phenalen-1-yl, phenalen-2-yl, phenanthren-1-yl, phenanthren-2-yl, phenanthren-3-yl, phenanthren-4-yl, phenanthren-9-yl, anthracen-1-yl, anthracen-2-yl, anthracen-9-yl, and the substitution products thereof, and preferably a group selected from the group consisting of benzen-1-yl (phenyl group), naphthalen-1-yl, naphthalen-2-yl, and the substitution products thereof.

By using the nucleic acids and photocrosslinking agent according to the present invention, nucleic acids can be linked without the use of an enzyme and under relaxed conditions within a short period of time compared to the conventional cases. In addition, the nucleic acids and photocrosslinking agent according to the present invention can be easily obtained, compared to conventional photocrosslinking agents, by carrying out chemical modifications for provision of photocrosslinking properties, labeled sites for detection, and the like, and which include structures capable of easily obtaining derivatives in which the modifications depending on the intended use have been made.

Such excellent properties of the photocrosslinking agent according to the present invention have been achieved as a result of a direct provision of a triazole structure to a vinyl group as well as a provision of a substituent which can be conjugated with the triazole structure.

In addition, the present invention also provides a production method of the nucleic acids used as a photocrosslinking agent, and the nucleic acids, an organic azidated product and a modifying agent which can be used for the production method.

Accordingly, the present invention also includes the following aspects [5] to [9].

[5] A production method of the nucleic acids according to the aspect [1], including a step of reacting nucleic acids which include a nucleic acid as well as a peptide nucleic acid, with an aromatic azidated product represented by the following formula X: the nucleic acids including a group represented by the following formulae VI, VII, VIII, or IX as a nucleobase:

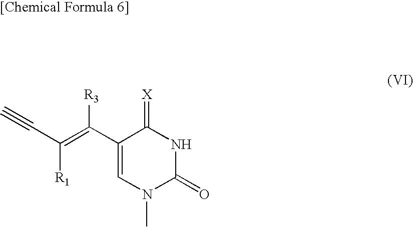

(with the proviso that in the formula VI, X represents O, S or NH;

each of R₁ and R₃ independently represents hydrogen, an alkyl group of 1 to 6 carbon atoms, an alkoxy group of 1 to 6 carbon atoms, a cyano group, or an acyl group of 1 to 6 carbon atoms);

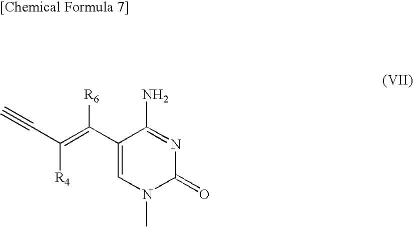

(with the proviso that in the formula VII, each of R₄ and R₆ independently represents hydrogen, an alkyl group of 1 to 6 carbon atoms, an alkoxy group of 1 to 6 carbon atoms, a cyano group, or an acyl group of 1 to 6 carbon atoms);

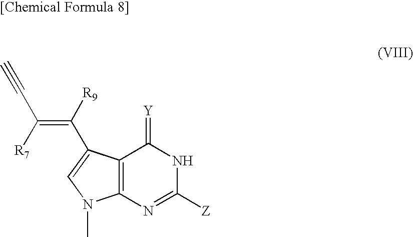

(with the proviso that in the formula VIII, Y represents O, S or NH;

Z represents NH₂ when Y represents O or S, and represents a hydrogen atom when Y represents NH;

each of R₇ and R₉ independently represents hydrogen, an alkyl group of 1 to 6 carbon atoms, an alkoxy group of 1 to 6 carbon atoms, a cyano group, or an acyl group of 1 to 6 carbon atoms);

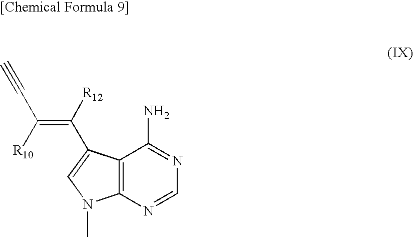

(with the proviso that in the formula IX, each of R₁₀ and R₁₂ independently represents hydrogen, an alkyl group of 1 to 6 carbon atoms, an alkoxy group of 1 to 6 carbon atoms, a cyano group, or an acyl group of 1 to 6 carbon atoms);

[Formula 1]

Ra—N₃ (X)

(with the proviso that in the formula X, Ra represents a substituent which can be conjugated with a triazole structure).

[6] Nucleic acids which include a nucleic acid as well as a peptide nucleic acid, the nucleic acids including a group represented by the following formulae VI, VII, VIII, or IX as a nucleobase:

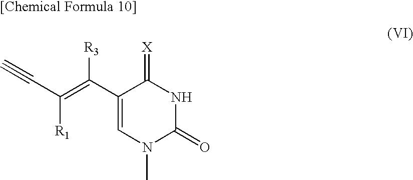

(with the proviso that in the formula VI, X represents O, S or NH;

each of R₁ and R₃ independently represents hydrogen, an alkyl group of 1 to 6 carbon atoms, an alkoxy group of 1 to 6 carbon atoms, a cyano group, or an acyl group of 1 to 6 carbon atoms);

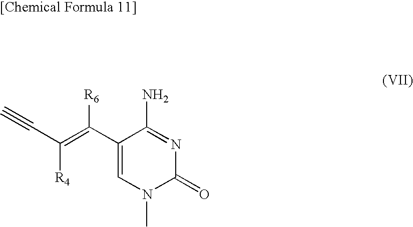

(with the proviso that in the formula VII, each of R₄ and R₆ independently represents hydrogen, an alkyl group of 1 to 6 carbon atoms, an alkoxy group of 1 to 6 carbon atoms, a cyano group, or an acyl group of 1 to 6 carbon atoms);

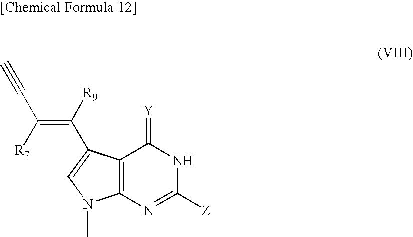

(with the proviso that in the formula VIII, Y represents O, S or NH;

Z represents NH₂ when Y represents O or S, and represents a hydrogen atom when Y represents NH;

each of R₇ and R₉ independently represents hydrogen, an alkyl group of 1 to 6 carbon atoms, an alkoxy group of 1 to 6 carbon atoms, a cyano group, or an acyl group of 1 to 6 carbon atoms);

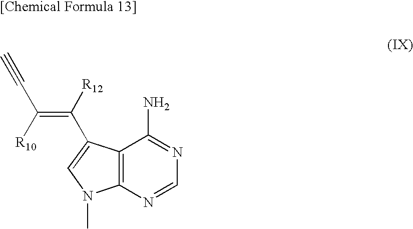

(with the proviso that in the formula IX, each of R₁₀ and R₁₂ independently represents hydrogen, an alkyl group of 1 to 6 carbon atoms, an alkoxy group of 1 to 6 carbon atoms, a cyano group, or an acyl group of 1 to 6 carbon atoms).

[7] An aromatic azidated product represented by the following formula X:

[Formula 2]

Ra—N₃ (X)

(with the proviso that in the formula X, Ra represents a substituent which can be conjugated with a triazole structure).

[8] A modifying agent for nucleic acids including the aromatic azidated product according to the aspect [7].

[9] A method for modifying nucleic acids using the aromatic azidated product according to the aspect [7].

By using the production method according to the present invention, nucleic acids serving as a photocrosslinking agent can be obtained by conducting a reaction under relaxed conditions to thereby form a triazole structure. Since the reaction conditions are considerably relaxed conditions compared to the reaction conditions typically employed in the organic synthesis, the organic substituent prepared as an organic azidated product can be added to the triazole structure extremely rapidly and also simply. For this reason, by using the production method according to the present invention, the chemical modifications for providing high photocrosslinking properties, labeled sites for detection, and the like, can easily be carried out to thereby obtain photocrosslinkable nucleic acids, and the derivatives in which a substituent is introduced depending on the intended use can easily be obtained as the photocrosslinkable nucleic acids.

Accordingly, the present invention also includes the following aspects [10] to [13].

[10] The production method according to the aspect [5], wherein Ra is a monovalent group of an aromatic group to which a labeled site is provided.

[11] The aromatic azidated product according to the aspect [7], wherein Ra is a monovalent group of an aromatic group to which a labeled site is provided.

[12] A labeled site introducing agent for nucleic acids, including the aromatic azidated product according to the aspect [11].

[13] A method for introducing a labeled site to nucleic acids using the aromatic azidated product according to the aspect [11].

In addition, the present invention also includes photocrosslinking property strengthening agent for photocrosslinkable nucleic acids including the aromatic azidated product according to the aspect [11], and also includes a method for strengthening the photocrosslinking properties of the photocrosslinkable nucleic acids using the aromatic azidated product according to the aspect [11].

Further, the present invention also includes the following aspects [14] to [17].

[14] Use of the nucleic acids according to any one of the aspects [1] and [2] for a photocrosslinking process.

[15] Nucleic acids which include a nucleic acid as well as a peptide nucleic acid, the nucleic acids including a triazole structure produced by the production method according to the aspect [5] or [10].

[16] Use of the aromatic azidated product according to the aspect [7] for modifying nucleic acids.

[17] Use of the aromatic azidated product according to the aspect [11] for introducing a labeled site to nucleic acids.

In addition, the present invention also includes the use of the aromatic azidated product according to the aspect [11] for strengthening the photocrosslinking properties of the photocrosslinkable nucleic acids.

The present invention also includes a compound analogous to nucleic acid base having a group represented by the formulae I, III, IV, or V, also includes a nucleoside or a derivative thereof which has a group represented by the formulae I, III, IV, or V, and also includes a nucleotide or a derivative thereof which has a group represented by the formulae I, III, IV, or V. These compounds are useful in synthesizing the nucleic acids according to the present invention, and they themselves are provided with photoresponsiveness.

Accordingly, the present invention also includes the following aspects [18] to [21].

[18] A compound formed by a group represented by the formulae I, III, IV, or V, and hydrogen, bonded therein.

[19] A nucleoside and a derivative thereof including a group represented by the formulae I, III, IV, or V as a nucleobase.

[20] A nucleotide and a derivative thereof including a group represented by the formulae I, III, IV, or V as a nucleobase.

[21] A compound formed by a group represented by the formulae I, II, IV, or V, and a group represented by the following formula XI or XII, bonded therein:

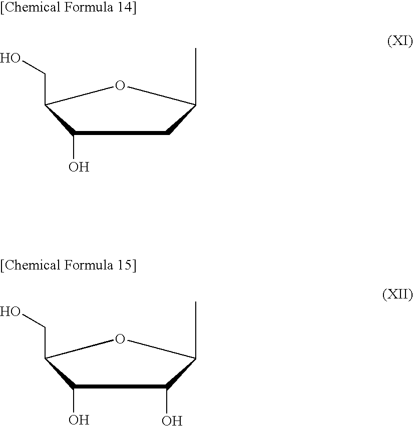

The present invention also includes a compound analogous to nucleic acid base having a group represented by the following formulae VI, VII, VIII, or IX, also includes a nucleoside or a derivative thereof which has a group represented by the formulae VI, VII, VIII, or IX, and also includes a nucleotide or a derivative thereof which has a group represented by the formulae VI, VII, VIII, or IX. These compounds are useful in synthesizing the nucleic acids according to the present invention.

Accordingly, the present invention also includes the following aspects [22] to [23].

[23] A compound formed by a group represented by the formulae VI, VII, VIII, or IX, and hydrogen, bonded therein.

[24] A nucleoside and a derivative thereof including a group represented by the following formulae VI, VII, VIII, or IX as a nucleobase.

[25] A nucleotide and a derivative thereof including a group represented by the following formulae VI, VII, VIII, or IX as a nucleobase:
[26] A compound formed by a group represented by the formulae VI, VII, VIII, or IX, and a group represented by the following formula XI or XII, bonded therein:

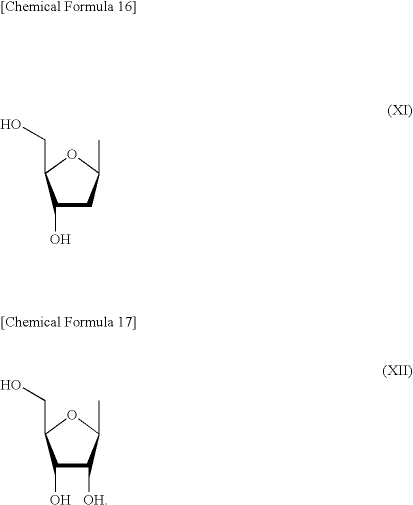

In the group represented by the formulae VI, VII, VIII, or IX, an ethynyl group which corresponds to the triple bond moiety may be protected by a protecting group. Examples of the protecting group include a trimethylsilyl (TMS) group.

Accordingly, the present invention also includes the following aspects [27] to [32].

[27] The method according to the aspect [5], wherein in the group represented by the formulae VI, VII, VIII, or IX, a protecting group is added to an ethynyl group.

[28] The nucleic acids according to the aspect [6], wherein in the group represented by the formulae VI, VII, VIII, or IX, a protecting group is added to an ethynyl group.

[29] The compound according to the aspect [23], wherein in the group represented by the formulae VI, VII, VIII, or IX, a protecting group is added to an ethynyl group.

[30] The nucleoside and the derivative thereof according to the aspect [24], wherein in the group represented by the formulae VI, VII, VIII, or IX, a protecting group is added to an ethynyl group.

[31] The nucleotide and the derivative thereof according to the aspect [25], wherein in the group represented by the formulae VI, VII, VIII, or IX, a protecting group is added to an ethynyl group.

[32] The compound according to the aspect [26], wherein in the group represented by the formulae VI, VII, VIII, or IX, a protecting group is added to an ethynyl group.

Further, the present invention also includes the following aspects [33] to [36].

[33] The nucleic acids including Ra according to the aspect [2], wherein an aromatic substituent is a phenyl group.

[34] The nucleic acids including Ra according to the aspect [2], wherein an aromatic substituent is a methoxyphenyl group.

[35] The nucleic acids including Ra according to the aspect [2], wherein an aromatic substituent is a cyanophenyl group.

[36] The nucleic acids including Ra according to the aspect [2], wherein an aromatic substituent is a naphthalene group.

According to the present invention, nucleic acids can be linked without the use of an enzyme and under relaxed conditions within a short period of time compared to the conventional cases. For example, the linking which required a reaction time of a few minutes to a few tens of minutes in the conventional photocrosslinking technique can be carried out within a reaction time of a mere few seconds to a few tens of seconds.

In addition, according to the present invention, since a photocrosslinking agent can be obtained by conducting a reaction under extremely relaxed conditions and thereby to form a triazole structure, modifications such as the addition of a labeled site for detection can be easily conducted, and the derivatives in which a substituent is introduced depending on the intended use can be easily obtained. As a result, the photocrosslinking agent according to the present invention can be used for an extremely wide range of applications.

### BRIEF DESCRIPTION OF THE DRAWINGS

FIG. 1 is a chromatogram showing the conversion from ODN including ^{EV}U to ODN (^{BTV}U) using benzylazido;

FIG. 2 is a chromatogram showing the conversion from ODN including ^{EV}U to ODN (^{PTV}U) using 1-azidobenzene;

FIG. 3 is a chromatogram showing the conversion from ODN including ^{EV}U to ODN (^{MPTV}U) using p-anisidine;

FIG. 4 is a chromatogram showing the conversion from ODN including ^{EV}U to ODN (^{CPTV}U) using 1-azidobenzonitrile;

FIG. 5 is a chromatogram showing the conversion from ODN including ^{EV}U using 1-naphthylazido;

FIG. 6 is a chromatogram showing the photolinkage of ODN (^{BTV}U);

FIG. 7 is a chromatogram showing the photolinkage of ODN (^{PTV}U)

FIG. 8 is a chromatogram showing the photolinkage of ODN (^{MPTV}U);

FIG. 9 is a chromatogram showing the photolinkage of ODN (^{CPTV}U);

FIG. 10 is a chromatogram showing the photolinkage of ODN (^{NPTV}U);

FIG. 11 is a chromatogram showing the photolinkage of ODN (^{CV}U); and

FIG. 12 is a graph in which the progresses of photolinkages over time using photocrosslinkable nucleic acids are compared.

### DETAILED DESCRIPTION OF THE INVENTION

The present invention will be described in detail by the following specific embodiment. The present invention is not limited to the specific embodiment shown below as an example.

The present invention is characterized by nucleic acids which include a nucleic acid as well as a peptide nucleic acid, the nucleic acids including a group represented by the following formulae I, III, IV, or V as a nucleobase:

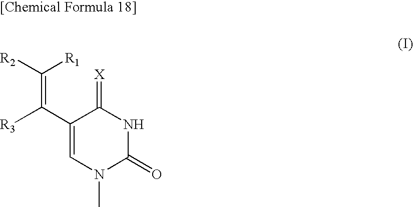

(with the proviso that in the formula I, X represents O, S or NH;

each of R₁ and R₃ independently represents hydrogen, an alkyl group of 1 to 6 carbon atoms, an alkoxy group of 1 to 6 carbon atoms, a cyano group, or an acyl group of 1 to 6 carbon atoms;

and R₂ is a group represented by the following formula II:

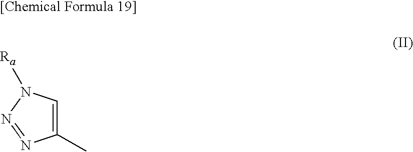

(with the proviso that in the formula II, Ra represents a substituent which can be conjugated with a triazole structure));

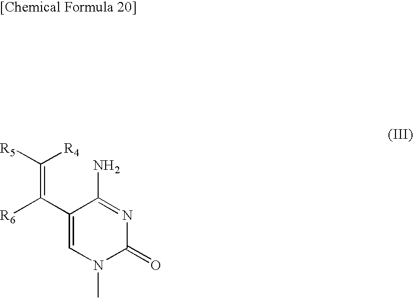

(with the proviso that in the formula III, each of R₄ and R₆ independently represents hydrogen, an alkyl group of 1 to 6 carbon atoms, an alkoxy group of 1 to 6 carbon atoms, a cyano group, or an acyl group of 1 to 6 carbon atoms;

and R₅ is a group represented by the formula II (with the proviso that in the formula II, Ra represents a substituent which can be conjugated with a triazole structure));

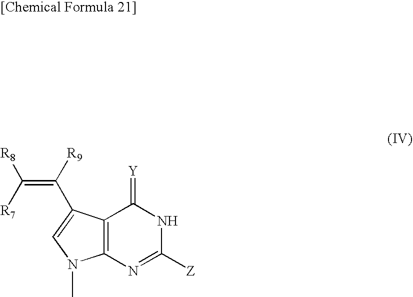

(with the proviso that in the formula IV, Y represents O, S or NH;

Z represents NH₂ when Y represents O or S, and represents a hydrogen atom when Y represents NH;

each of R₇ and R₉ independently represents hydrogen, an alkyl group of 1 to 6 carbon atoms, an alkoxy group of 1 to 6 carbon atoms, a cyano group, or an acyl group of 1 to 6 carbon atoms;

and R₈ is a group represented by the following formula II (with the proviso that in the formula II, Ra represents a substituent which can be conjugated with a triazole structure));

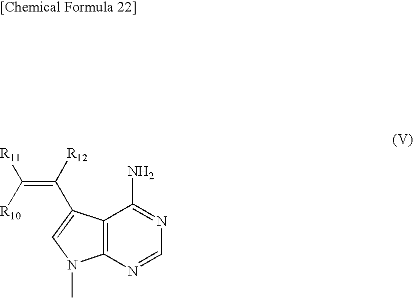

(with the proviso that in the formula V, each of R₁₀ and R₁₂ independently represents hydrogen, an alkyl group of 1 to 6 carbon atoms, an alkoxy group of 1 to 6 carbon atoms, a cyano group, or an acyl group of 1 to 6 carbon atoms;

and R₁₁ is a group represented by the following formula II (with the proviso that in the formula II, Ra represents a substituent which can be conjugated with a triazole structure)).

By using the nucleic acids according to the present invention as a photocrosslinking agent, nucleic acids can be linked without the use of an enzyme and under relaxed conditions within a short period of time compared to the conventional cases. In addition, the above-mentioned nucleic acids have a structure which enables the modifications, such as the addition of a labeled site for detection, and also an easy obtaining of a derivative in which a modification depending on the intended use has been introduced.

Such excellent properties of the photocrosslinking agent according to the present invention have been achieved as a result of a direct provision of a triazole structure to a vinyl group as well as a provision of a substituent which can be conjugated with the triazole structure.

The groups R₂, R₅, R₈ and R₁₁ are groups represented by the above-mentioned formula II and include a triazole structure and a substituent Ra added so as to be able to conjugate with the triazole structure.

As the substituent Ra, any substituent can be used as long as the substituent is capable of conjugating with the triazole structure to be added. Examples of such substituent which can be conjugated include a monovalent group of an aromatic compound, and more specific examples thereof include a monovalent group formed as a result of the loss of one hydrogen atom from the ring of the aromatic compound. Examples of the aromatic compound which can be used by being converted into a monovalent group include an aromatic group having 1, 2, 3 or more rings. The aromatic compounds which can be used include monocyclic aromatic compounds having one ring and condensed polycyclic aromatic compounds having 2 or more condensed rings. In the present invention, although the number of rings included in the aromatic compound is not limited, it is typically within a range from 1 to 10, preferably from 1 to 8, more preferably from 1 to 6, still more preferably from 1 to 4, and particularly preferably from 1 to 3. The greater the number of rings included in the aromatic compound, the stronger the extent of conjugation with the triazole structure. However, the possibility of the occurrence of steric hindrance at the time of linkage of nucleic acids becomes higher as the number of rings increases. In the present invention, although the aromatic ring is not limited, it is formed typically from a 4 to 8-membered ring, preferably from a 4 to 7-membered ring, more preferably from a 4 to 6-membered ring, still more preferably from a 5 to 6-membered ring, and particularly preferably from a 6-membered ring.

Among the aromatic compounds which can be used by being converted into a monovalent group, examples of the monocyclic aromatic compound include benzene and the substitution products thereof. Examples of the substitution products include the substitution products in which 1, 2, 3 or more hydrogen atoms, preferably 1 or 2 hydrogen atoms, and more preferably 1 hydrogen atom of the ring is substituted, typically with an alkyl group of 1 to 6 carbon atoms, an alkoxy group of 1 to 6 carbon atoms or a cyano group, preferably with an alkyl group of 1 to 3 carbon atoms, an alkoxy group of 1 to 3 carbon atoms and/or a cyano group, and particularly preferably with a methyl group, an ethyl group, a methoxy group, an ethoxy group, and/or a cyano group. Preferable examples of the monovalent group of a monocyclic aromatic compound include a substituted or unsubstituted phenyl (benzen-1-yl) group. According to the present invention, even a derivative in which the type and position of the substituent is changed (i.e., a substituted phenyl group) can be suitably used for the photocrosslinking reaction.

Among the aromatic compounds which can be used by being converted into a monovalent group, examples of the condensed polycyclic aromatic compounds include pentalene, indene, naphthalene, azulene, heptalene, biphenylene, as-indacene, s-indacene, acenaphthylene, fluorene, phenalene, phenanthrene, anthracene, fluoranthene, acephenantolylene, aceantolylene, triphenylene, pyrene, chrysene, tetracene (naphthacene), pleiadene, picene, perylene, pentaphene, pentacene, tetraphenylene, hexaphene, hexacene, rubicene, coronene, trinaphthylene, heptaphene, heptacene, pyranthrene, obalene and hexahelicene; heterocyclic aromatic compounds such as thiophene, thianthrene, furan, 2H-pyran, isobenzofuran, isochromene, 4H-chromene, xanthene, phenoxathine, pyrrole, 2H-pyrrole, imidazole, pyrazole, isothiazole, isoxazole, pyridine, pyrazine, pyrimidine, pyridazine, 1H-pyrrolidine, indolizine, isoindole, indole, indazole, purine, 4H-quinolidine, isoquinoline, quinoline, phthalazine, naphthylidine, quinoxaline, quinazoline, cinoline, pteridine, carbazole, β-carboline, phenanthredine, acridine, perimidine, phenanthroline, phenazine, phenothiazine and phenoxazine; and the substitution products thereof. Examples of the substitution products include the substitution products in which 1, 2, 3 or more hydrogen atoms, preferably 1 or 2 hydrogen atoms, and more preferably 1 hydrogen atom of the ring is substituted, typically with an alkyl group of 1 to 6 carbon atoms, an alkoxy group of 1 to 6 carbon atoms or a cyano group, preferably with an alkyl group of 1 to 3 carbon atoms, an alkoxy group of 1 to 3 carbon atoms and/or a cyano group, and particularly preferably with a methyl group, an ethyl group, a methoxy group, an ethoxy group, and/or a cyano group.

Preferable examples of the monovalent group of condensed polycyclic aromatic compound include a pentalen-1-yl group, a pentalen-2-yl group, a pentalen-3-yl group, an inden-2-yl group, an inden-3-yl group, an inden-4-yl group, an inden-5-yl group, an inden-6-yl group, an inden-7-yl, a naphthalen-1-yl group, a naphthalen-2-yl group, an azulen-1-yl group, an azulen-2-yl group, an azulen-3-yl group, an azulen-4-yl group, an azulen-5-yl group, an azulen-6-yl group, an azulen-7-yl group, an azulen-8-yl group, a heptalen-1-yl group, a heptalen-2-yl group, a heptalen-3-yl group, a biphenylen-1-yl group, a biphenylen-2-yl group, an as-indacen-1-yl group, an as-indacen-2-yl group, an as-indacen-3-yl group, an as-indacen-4-yl group, an as-indacen-5-yl group, an as-indacen-6-yl group, an as-indacen-7-yl group, an as-indacen-8-yl group, a s-indacen-1-yl group, a s-indacen-2-yl group, a s-indacen-3-yl group, a s-indacen-4-yl group, an acenaphthylen-1-yl group, an acenaphthylen-3-yl group, an acenaphthylen-4-yl group, an acenaphthylen-5-yl group, a fluoren-1-yl group, a fluoren-2-yl group, a fluoren-3-yl group, a fluoren-4-yl group, a phenalen-1-yl group, a phenalen-2-yl group, a phenanthren-1-yl group, a phenanthren-2-yl group, a phenanthren-3-yl group, a phenanthren-4-yl group, a phenanthren-5-yl group, a phenanthren-6-yl group, a phenanthren-7-yl group, a phenanthren-8-yl group, a phenanthren-9-yl group, an anthracen-1-yl group, an anthracen-2-yl group, an anthracen-9-yl group, a fluoranthen-1-yl group, a fluoranthen-2-yl group, a fluoranthen-3-yl group, a fluoranthen-4-yl group, a fluoranthen-9-yl group, a fluoranthen-10-yl group, an acephenantolylen-1-yl group, an acephenantolylen-2-yl group, an acephenantolylen-3-yl group, an acephenantolylen-4-yl group, an acephenantolylen-5-yl group, an acephenantolylen-6-yl group, an acephenantolylen-7-yl group, an acephenantolylen-8-yl group, an acephenantolylen-9-yl group, an acephenantolylen-10-yl group, an aceantolylen-1-yl group, an aceantolylen-2-yl group, an aceantolylen-3-yl group, an aceantolylen-4-yl group, an aceantolylen-5-yl group, an aceantolylen-6-yl group, an aceantolylen-7-yl group, an aceantolylen-8-yl group, an aceantolylen-9-yl group, an aceantolylen-10-yl group, a triphenylen-1-yl group, a triphenylen-2-yl group, a pyren-1-yl group, a pyren-2-yl group, a chrysen-1-yl group, a chrysen-2-yl group, a chrysen-3-yl group, a chrysen-4-yl group, a chrysen-5-yl group, a chrysen-6-yl group, a tetracen-1-yl (naphthacen-1-yl) group, a tetracen-2-yl (naphthacen-2-yl) group, a tetracen-5-yl (naphthacen-5-yl) group, and the substitution products thereof; preferably a pentalen-1-yl group, a pentalen-2-yl group, a pentalen-3-yl group, an inden-2-yl group, an inden-3-yl group, an inden-4-yl group, an inden-5-yl group, an inden-6-yl group, an inden-7-yl, a naphthalen-1-yl group, a naphthalen-2-yl group, an azulen-1-yl group, an azulen-2-yl group, an azulen-3-yl group, an azulen-4-yl group, an azulen-5-yl group, an azulen-6-yl group, an azulen-7-yl group, an azulen-8-yl group, a heptalen-1-yl group, a heptalen-2-yl group, a heptalen-3-yl group, a biphenylen-1-yl group, a biphenylen-2-yl group, an as-indacen-1-yl group, an as-indacen-2-yl group, an as-indacen-3-yl group, an as-indacen-4-yl group, an as-indacen-5-yl group, an as-indacen-6-yl group, an as-indacen-7-yl group, an as-indacen-8-yl group, a s-indacen-1-yl group, a s-indacen-2-yl group, a s-indacen-3-yl group, a s-indacen-4-yl group, an acenaphthylen-1-yl group, an acenaphthylen-3-yl group, an acenaphthylen-4-yl group, an acenaphthylen-5-yl group, a fluoren-1-yl group, a fluoren-2-yl group, a fluoren-3-yl group, a fluoren-4-yl group, a phenalen-1-yl group, a phenalen-2-yl group, a phenanthren-1-yl group, a phenanthren-2-yl group, a phenanthren-3-yl group, a phenanthren-4-yl group, a phenanthren-9-yl group, an anthracen-1-yl group, an anthracen-2-yl group, an anthracen-9-yl group, and the substitution products thereof; and particularly preferably a naphthalen-1-yl group, a naphthalen-2-yl group, and the substitution products thereof. According to the present invention, even a derivative in which the type and position of the substituent is changed (i.e., a monovalent group of a condensed polycyclic aromatic compound) can be suitably used for the photocrosslinking reaction. As a monovalent group of a condensed polycyclic aromatic compound which is particularly preferable, a substituted or unsubstituted naphthalen-1-yl group or naphthalen-2-yl group, and the substitution products thereof can be mentioned. As described above, examples of the substitution products include the substitution products in which 1, 2, 3 or more hydrogen atoms, preferably 1 or 2 hydrogen atoms, and more preferably 1 hydrogen atom of the ring is substituted, typically with an alkyl group of 1 to 6 carbon atoms, an alkoxy group of 1 to 6 carbon atoms, a cyano group and/or an acyl group of 1 to 6 carbon atoms, preferably with an alkyl group of 1 to 3 carbon atoms, an alkoxy group of 1 to 3 carbon atoms and/or a cyano group, and particularly preferably with a methyl group, an ethyl group, a methoxy group, an ethoxy group, and/or a cyano group.

The greater the extent of conjugation of the substituent Ra with the triazole structure to be added, the greater the extent of photoresponsiveness of the nucleic acids according to the present invention, thereby increasing the photocrosslinking rate. When a monovalent group of an aromatic compound is used as the substituent Ra, the more the number of conjugated rings, the greater the extent of photoresponsiveness, thereby increasing the photocrosslinking rate. With respect to the presence/absence and type of substituent in those cases where the hydrogen atom of a conjugated ring, the greater the extent of conjugation with the triazole structure, the greater the contribution thereof to the increases of photoresponsiveness and photocrosslinking rate. However, the contribution of the number of conjugated rings to the increases of photoresponsiveness and photocrosslinking rate is greater than that of the presence/absence and type of substituent in the conjugated ring.

In the preferred embodiment of the present invention, the nucleic acids according to the present invention have a group represented by the formula I as a nucleobase. In the preferred embodiment of the present invention, X in the formula I is O. In other words, the nucleobase (base moiety) represented by the formula I is a uracil derivative or a thymine derivative.

In the preferred embodiment of the present invention, the nucleic acids according to the present invention have a group represented by the formula III as a nucleobase. In other words, the base moiety represented by the formula III is a cytosine derivative.

In the preferred embodiment of the present invention, the nucleic acids according to the present invention have a group represented by the formula IV as a nucleobase. In the preferred embodiment of the present invention, Y in the formula IV is O while Z is NH₂. In other words, the base moiety represented by the formula IV is a guanine derivative.

In the preferred embodiment of the present invention, the nucleic acids according to the present invention have a group represented by the formula V as a nucleobase. In other words, the base moiety represented by the formula V is an adenine derivative.

Each of R₁, R₃, R₄, R₆, R₇, R₉, R₁₀ and R₁₂ independently represents hydrogen, an alkyl group, an alkoxy group, a cyano group, or an acyl group of 1 to 6 carbon atoms. Suitable examples of the alkyl group include alkyl groups typically having 1 to 8 carbon atoms, preferably having 1 to 6 carbon atoms, more preferably having 1 to 5 carbon atoms, still more preferably having 1 to 4 carbon atoms, still more preferably having 1 to 3 carbon atoms, still more preferably having 1 to 2 carbon atoms, and still more preferably having 1 carbon atom. Suitable examples of the alkoxy group include alkoxy groups typically having 1 to 8 carbon atoms, preferably having 1 to 6 carbon atoms, more preferably having 1 to 5 carbon atoms, still more preferably having 1 to 4 carbon atoms, still more preferably having 1 to 3 carbon atoms, still more preferably having 1 to 2 carbon atoms, and still more preferably having 1 carbon atom. Suitable examples of the acyl group include acyl groups typically having 1 to 8 carbon atoms, preferably having 1 to 6 carbon atoms, more preferably having 1 to 5 carbon atoms, still more preferably having 1 to 4 carbon atoms, still more preferably having 1 to 3 carbon atoms, still more preferably having 1 to 2 carbon atoms, and still more preferably having 1 carbon atom.

In the particularly preferable embodiment, each of R₁, R₃, R₄, R₆, R₇, R₉, R₁₀ and R₁₂ independently represents hydrogen, a methyl group, an ethyl group, a methoxy group, an ethoxy group, a cyano group, or an acetyl group, and preferably represents hydrogen, a methyl group, a methoxy group or a cyano group.

The term nucleic acids used in the present invention includes a nucleic acid and a peptide nucleic acid (PNA), and even includes a mononucleotide. The nucleic acid includes DNA and RNA which are natural nucleic acids, and also includes modified nucleic acids, such as LNA (ENA), which are non-natural (artificial) nucleic acids.

The nucleic acids according to the present invention include a base exhibiting a high level of photoresponsiveness due to the addition of a triazole structure, to which a substituent capable of conjugation is added, to a vinyl group, and thus can be photolinked with another nucleic acid (nucleic acids) by the irradiation of light. In other words, the nucleic acids according to the present invention are photoresponsive nucleic acids as well as photocrosslinkable nucleic acids, and thus can be used as photocrosslinking agents.

Examples of another nucleic acid (nucleic acids) which can be photolinked with the nucleic acids according to the present invention include a nucleic acid (nucleic acids) having a pyrimidine ring as a nucleobase. Examples of the nucleic acid (nucleic acids) having a pyrimidine ring as a nucleobase include a nucleic acid (nucleic acids) having cytosine, uracil, thymine, and a derivative thereof as a nucleobase, and the nucleic acid (nucleic acids) preferably has cytosine, uracil or a derivative thereof, particularly preferably cytosine, as a nucleobase.

Light irradiated for the photocrosslinking process is preferably light typically having a wavelength within a range from 350 to 380 nm, preferably within a range from 360 to 370 nm, and more preferably 366 nm, and is particularly preferably laser light having a single wavelength of 366 nm.

The nucleic acid (nucleic acids) according to the present invention can undergo a photocleavage process, following the photolinkage with another nucleic acid (nucleic acids) by photoirradiation, by the irradiation of light having a different wavelength to that used at the time of photolinkage. In other words, the nucleic acids according to the present invention enable a reversible photocrosslinking process, and thus can be used as reversible photocrosslinking agents.

Light irradiated for the photocleavage process is preferably light typically having a wavelength within a range from 300 to 320 nm, preferably within a range from 305 to 315 nm, and more preferably 312 nm, and is particularly preferably laser light having a single wavelength of 312 nm.

Since the photolinkage and photocleavage according to the present invention are taking advantages of photoreactions, they are not particularly limited in terms of pH, temperature, salt concentration or the like, and thus can be carried out by the irradiation of light in a solution in which pH, temperature, and salt concentration are adjusted so that biopolymers such as nucleic acids are stably present.

Furthermore, the present invention also includes a production method of the nucleic acids according to the present invention by reacting nucleic acids, which include a nucleic acid as well as a peptide nucleic acid, the nucleic acids including a group represented by the following formula VI, formula VII, formula VIII, or formula IX as a nucleobase:

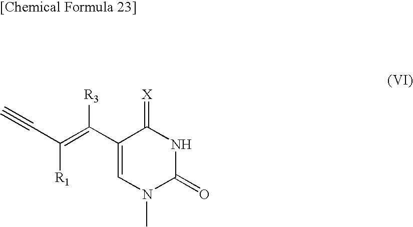

(with the proviso that in the formula VI, X represents O, S or NH;

each of R₁ and R₃ independently represents hydrogen, an alkyl group of 1 to 6 carbon atoms, an alkoxy group of 1 to 6 carbon atoms, a cyano group, or an acyl group of 1 to 6 carbon atoms);

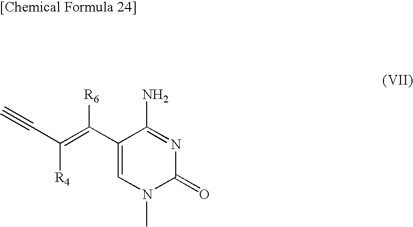

(with the proviso that in the formula VII, each of R₄ and R₆ independently represents hydrogen, an alkyl group of 1 to 6 carbon atoms, an alkoxy group of 1 to 6 carbon atoms, a cyano group, or an acyl group of 1 to 6 carbon atoms);

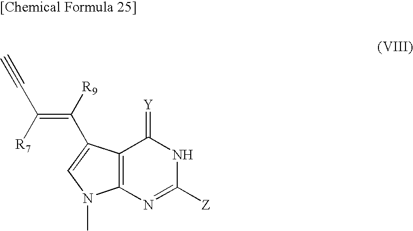

(with the proviso that in the formula VIII, Y represents O, S or NH;

Z represents NH₂ when Y represents O or S, and represents a hydrogen atom when Y represents NH;

each of R₇ and R₉ independently represents hydrogen, an alkyl group of 1 to 6 carbon atoms, an alkoxy group of 1 to 6 carbon atoms, a cyano group, or an acyl group of 1 to 6 carbon atoms);

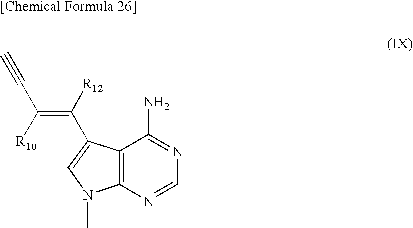

(with the proviso that in the formula IX, each of R₁₀ and R₁₂ independently represents hydrogen, an alkyl group of 1 to 6 carbon atoms, an alkoxy group of 1 to 6 carbon atoms, a cyano group, or an acyl group of 1 to 6 carbon atoms);

with an aromatic azidated product represented by the following formula X:

[Formula 3]

Ra—N₃ (X)

(with the proviso that in the formula X, Ra represents a substituent which can be conjugated with a triazole structure).

Ra is as described as above. In other words, Ra—N₃ is preferably an aromatic azidated product composed of a monovalent group of an aromatic compound and N₃, although it can be used as long as it is an organic azidated product composed of a substituent capable of conjugating with the triazole structure to be added. Those described above with respect to Ra are applicable to the usable monovalent group of an aromatic compound as well as to the preferable monovalent group of an aromatic compound.

By using the production method according to the present invention, nucleic acids serving as a photocrosslinking agent can be obtained by conducting a reaction under relaxed conditions to thereby form a triazole structure. Since the reaction conditions are considerably relaxed conditions compared to the reaction conditions typically employed in the organic synthesis, the organic substituent prepared as an organic azidated product can be added to the triazole structure extremely rapidly and also simply. This cycloaddition reaction is a reaction analogous to the Huisgen cyclization which is one of the reactions collectively known as click reactions, and proceeds with an extremely high yield without being effected by the presence of water or various functional groups. In other words, the reaction can be conducted either in a water-based solvent or in an organic solvent as long as the solvent (environment) allows the presence of nucleic acids. For this reason, by using the production method according to the present invention, the modifications for adding a labeled site for detection and the like can easily be carried out, and the derivatives in which the substituent Ra is introduced depending on the intended use can easily be obtained as the photocrosslinkable nucleic acids.

As already stated, when the substituent Ra is a monovalent group of an aromatic compound, since the extent of the conjugated system (i.e., the number of conjugated rings) affects photoresponsiveness (photocrosslinking properties) far greater than the presence/absence and type of substituent in the conjugated ring, by selecting an adequate number of conjugate rings in advance, it is possible to add a desired labeled site by substituting the hydrogen in the conjugated ring while sufficiently maintaining the level of photoresponsiveness (photocrosslinking properties). In other words, the nucleic acids according to the present invention can easily undergo modifications, such as the addition of a labeled site for detection, by the production method according to the present invention, the nucleic acids and production method according to the present invention can be used in a wide range of applications.

As a labeled site to be introduced by addition, a known molecule or group can be used, and examples thereof include a fluorochrome, biotin, hapten, a peptide, a protein, an enzyme, ferrocene and a spin active compound.

### EXAMPLES

The present invention will be described in detail below using some Examples. The present invention is not limited to the following Examples.

### Synthesis of Photocrosslinkable Nucleic Acids

### Synthesis of ODN including 5-ethynylvinyl-2′-deoxyuridine (^{EV}U

The synthesis was conducted according to the following Scheme 1. It should be noted that the numbers attached to the compounds may be used in the following descriptions.

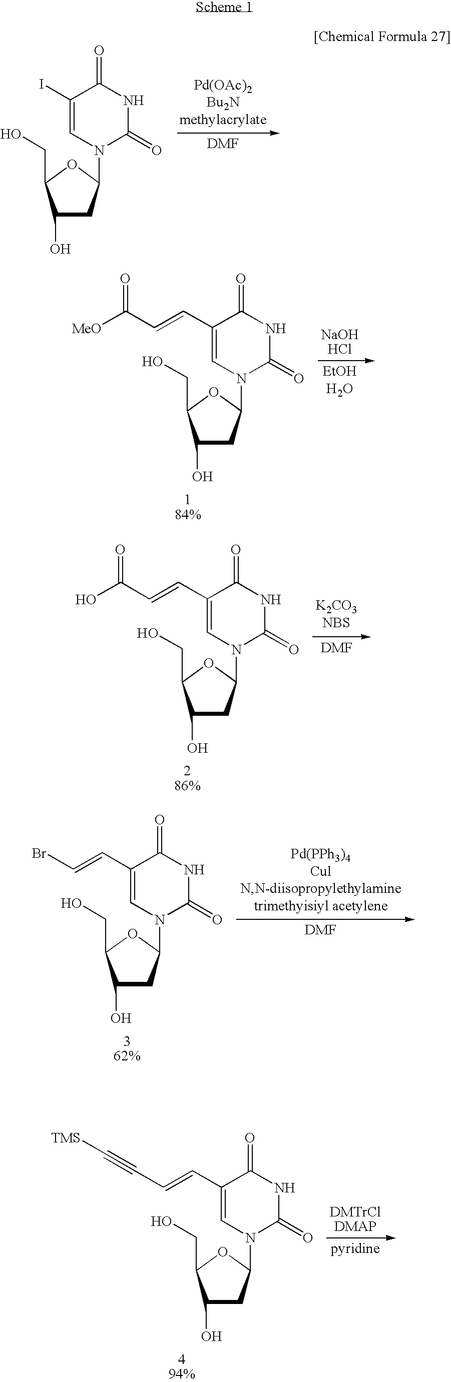

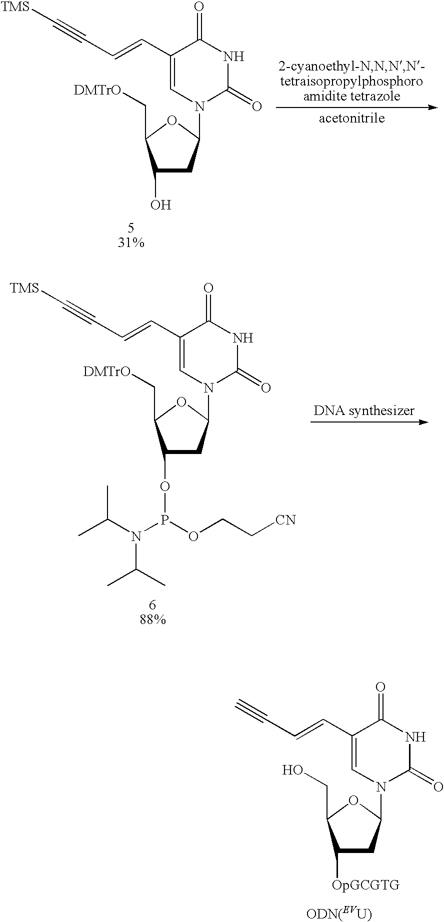

### (E)-5-(2-Carbomethoxyvinyl)-2′-deoxyuridine (1)

A 7 mL tube manufactured by Discover was charged with 1.00 g of 5-Iodo-deoxyuridine (3.20 mmol), followed by the addition of 0.07 g of Pd(OAc)₂ (0.32 mmol) and 3 mL of DMF thereto, and a nitrogen substitution process was then carried out twice. Subsequently, 0.76 mL of Bu₃N (3.20 mmol) and 0.43 mL of methylacrylate (4.80 mmol) were added thereto, and the resultant was then subjected to a microwave irradiation at 100° C. for 4 minutes while being stirred. This operation was repeated 4 times in total, and the resulting precipitates were removed by filtration. The resultant was subjected to a column purification, thereby yielding a compound 1 in the form of a white powder. The amount obtained was 3.36 g, which corresponded to a yield of 84%. ¹H-NMR (DMSO-d6) δ11.64 (bs, 1H), 8.41 (s, 1H), 7.36 (d, 1H, J=15.8 Hz), 6.84 (d, 1H, J=15.8 Hz), 6.12 (t, 1H, J=6.5 Hz), 5.25 (d, 1H, J=4.2 Hz), 5.16 (t, 1H, J=5.1 Hz), 4.24 (m, 1H), 3.79 (m, 1H), 3.67 (s, 3H), 3.64-3.54 (m, 2H), 2.17 (m, 2H).

### (E)-5-(2-Carboxyvinyl)-2′-deoxyuridine (2)

3M NaOH was added to 3.25 g (10.43 mmol) of the compound 1 until the compound 1 was dissolved therein, and the resultant was stirred at room temperature for 3 hours. After confirming the disappearance of the source material by TLC, 6 M HCl was gradually added to the resultant in an ice bath until the precipitates were formed. The resulting precipitates were collected, and then washed with hexane, thereby yielding a compound 2 in the form of a white powder. The amount obtained was 2.68 g, which corresponded to a yield of 86%. ¹H-NMR (DMSO-d₆) 611.61 (s, 1H), 8.37 (s, 1H), 7.28 (d, 1H, J=15.8 Hz), 6.12 (d, 1H, J=15.8 Hz), 6.12 (t, 1H, J=6.3 Hz), 5.20 (br, 1H), 5.18 (br, 1H, J=5.4 Hz), 4.25 (m, 1H), 3.79 (m, 1H), 3.59 (m, 2H), 2.18 (m, 2H).

### (E)-5-(2-Bromovinyl)-2′-deoxyuridine (3)

2.64 g (8.85 mmol) of the compound 2 was dissolved in 30 ml of DMF, followed by the addition of 1.84 g (13.28 mmol) of K₂CO₃ thereto, and the resultant was stirred at room temperature for 15 minutes. Then, 1.58 g (8.85 mmol) of N-Bromosuccinimide dissolved in 20 ml of DMF was gradually added thereto, followed by filtration, and the obtained precipitates were washed with DMF. The filtrate and cleaning liquid were collected, followed by the removal of solvents therefrom, and the resultant was then subjected to a column purification, thereby yielding a compound 3 in the form of a white powder. The amount obtained was 1.82 g, which corresponded to a yield of 62%. ¹H-NMR (DMSO) 611.56 (s, 1H), 8.06 (s, 1H), 7.22 (d, 1H, J=13.6 Hz), 6.83 (d, 1H, J=13.6 Hz), 6.11 (t, 1H, J=6.6 Hz), 5.27 (d, 1H, J=4.4 Hz), 5.10 (t, 1H, J=5.2 Hz), 4.23 (m, 1H), 3.77 (m, 1H), 2.12 (m, 2H).

### (E)-5-(2-trimethylsilyl ethynyl vinyl)-2′-deoxyuridine (4)

0.67 g (2.0 mmol) of the compound 3 was dissolved in 10 ml of DMF, followed by the addition of 0.23 g (0.2 mmol) of Pd (PPh₃)₄, 0.076 g (0.4 mmol) of CuI, and 1.7 ml (10 mmol) of N,N-diisopropylethylamine thereto, and the resultant was stirred for 10 minutes. Then, 0.83 ml (6.0 mmol) of trimethylsilyl acetylene was added thereto, and the resultant was stirred for 3 hours. After confirming the disappearance of the source material by TLC, solvents were removed, and the resultant was once dissolved in methanol, followed by filtration to collect the resulting precipitates. The resultant was subjected to a column purification, thereby yielding a compound 4. The amount obtained was 0.66 g, which corresponded to a yield of 94%. ¹H-NMR (DMSO) δ8.14 (s, 1H), 6.69 (d, 1H, J=16.2 Hz), 6.56 (d, 1H, J=16.2 Hz), 6.11 (t, 1H, J=6.3 Hz), 5.27 (d, 1H, J=4.4 Hz), 5.13 (t, 1H, J=5.2 Hz), 4.23 (m, 1H), 3.77 (m, 1H), 2.12 (m, 2H).

### 5′-O— (4,4′-Dimethoxytrityl)-(E)-5-(2-trimethylsilyl ethynyl vinyl)-2′-deoxyuridine (5)

0.60 g (1.7 mmol) of the compound 4 was charged into a 100 ml eggplant shaped flask, followed by a nitrogen substitution and pyridine azeotropy, and the resultant was dissolved in 10 ml of pyridine. Then, 0.70 g (2.0 mmol) of DMTrCl and 0.04 g (0.34 mmol) of DMAP were added thereto in an ice bath, and the resultant was stirred overnight. After confirming the disappearance of the source material by TLC, solvents were removed, and the resultant was purified by column purification (CHCl₃: MeOH=from 100:0 to 98:2) thereby yielding a compound 5. The amount obtained was 0.34 g, which corresponded to a yield of 31%. ¹H-NMR (CD₃OD) δ8.29 (s, 1H), 6.73 (d, 1H, J=16.2 Hz), 6.61 (d, 1H, J=16.2 Hz), 6.30 (t, 1H, J=6.6 Hz), 4.45 (m, 1H), 3.98 (m, 1H), 3.84 (m, 2H), 2.35 (m, 2H).

### 5′-O-(4,4′-Dimethoxytrityl)-(E)-5-(2-trimethylsilyl ethynyl vinyl)-2′-deoxyuridine phosphoroamidite (6)

0.2 g (0.31 mmol) of the compound 5 was charged into a rubber-sealed bottle, and an azeotropy with 2.0 ml of acetonitrile was carried out twice. Then 2.0 ml of acetonitrile, 0.097 ml (0.31 mmol) of 2-cyanoethyl-N,N,N′,N′-tetraisopropylphosphoroamidite, and 0.68 ml of 0.45 M tetrazole were added thereto, followed by stirring at room temperature for 2 hours. Subsequently, the resultant was extracted twice with ethyl acetate which underwent deacetylation, the solvents were removed therefrom, and the resultant was washed with Sat. NaHCO₃ aq. and H₂O. The organic phase was dried using MgSO₄ and the solvents were removed therefrom using an evaporator, followed by an azeotropy with 1.0 ml of acetonitrile, thereby yielding 0.23 g of a compound 6 (yield: 88%).

### Synthesis of ODN including ^{EV}U

An ODN (^{EV}U) containing ^{EV}U at the 5′ end thereof was synthesized twice in a 1 μmol scale using the ABI 3400 DNA synthesizer. After the excision from a solid-phase support using ammonia, 1 ml of a 28% aqueous ammonia solution was added thereto, and the resultant was incubated at 65° C. for 4 hours to conduct a deprotection, and was then freeze-dried. After the purification by HPLC using ammonium formate/acetonitrile (8 to 15%/30 min), the resultant was freeze-dried.

ODN(^{EV}U): 5′-^{EV}UGCGTG-3′.MALDI-TOF MS: Calcd. for ODN(^{EV}U) [(M+H)⁺] 1859.35, found 1859.41

### Synthesis of Aromatic Azidated Product

The following aromatic azidated products were synthesized, respectively, according to the following procedures. It should be noted that the numbers attached to the compounds may be used in the following descriptions.

### Synthesis of 1-azidobenzene

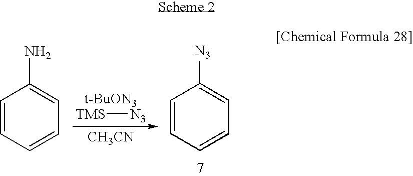

1.0 g (10.7 mmol) of aniline was charged into a 50 ml eggplant shaped flask, and was dissolved in 20 ml of acetonitrile, and 1.9 ml (16.1 mmol) of tert-butyl nitrite and 1.7 ml (12.8 mmol) of TMS-N₃ were added thereto in an ice bath, and the resultant was stirred for 1 hour. After confirming the disappearance of the source material as well as the presence of a product material by TLC, solvents were removed, and the resultant was subjected to a column purification, thereby yielding 0.55 g (4.7 mmol) of a compound 7. The yield was 43%. ¹H NMR (CDCl₃, 300 MHz) δ: 7.01 (d, J=7.6 Hz, 2H), 7.12 (t, J=7.6 Hz, 1H), 7.34 (t, J=7.6 Hz, 2H).

### Synthesis of 1-azido-4-methoxybenzene

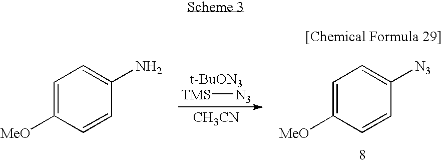

1.0 g (8.1 mmol) of p-anisidine was charged into a 50 ml eggplant shaped flask, and was dissolved in 10 ml of acetonitrile, and 1.45 ml (12.2 mmol) of tert-butyl nitrite and 1.30 ml (9.75 mmol) of TMS-N₃ were added thereto in an ice bath, and the resultant was stirred for 2 hours. After confirming the disappearance of the source material as well as the presence of a product material by TLC, solvents were removed, and the resultant was subjected to a column purification, thereby yielding 0.44 g (2.9 mmol) of a compound 8 in the form of yellow oil. The yield was 36%.

### Synthesis of 1-azidobenzonitrile

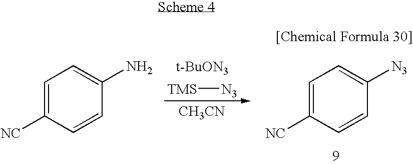

0.2 g (1.69 mmol) of 4-aminobenzonitrile was charged into a 20 ml eggplant shaped flask, and was dissolved in 4 ml of acetonitrile, and 0.3 ml (2.54 mmol) of tert-butyl nitrite and 0.27 ml (2.03 mmol) of TMS-N₃ were added thereto in an ice bath, and the resultant was stirred for 2 hours. After confirming the disappearance of the source material as well as the presence of a product material by TLC, solvents were removed, and the resultant was washed with cold hexane, thereby yielding 0.17 g (1.18 mmol) of a compound 9. The yield was 70%.

### Synthesis of 1-naphthylazido

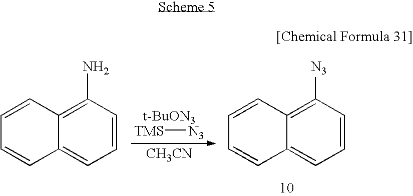

0.2 g (1.40 mmol) of 1-naphthylamine was charged into a 10 ml eggplant shaped flask, and was dissolved in 3 ml of acetonitrile, and 0.25 ml (2.10 mmol) of tert-butyl nitrite and 0.22 ml (1.68 mmol) of TMS-N₃ were added thereto in an ice bath, and the resultant was stirred for 2 hours. After confirming the progress of the reaction by TLC (hexane), solvents were removed, and the resultant was subjected to a column purification (hexane), thereby yielding 0.21 g (1.23 mmol) of a compound 10. The yield was 88%.

### Click Reaction of ODN Including ^{EV}U with Benzylazido

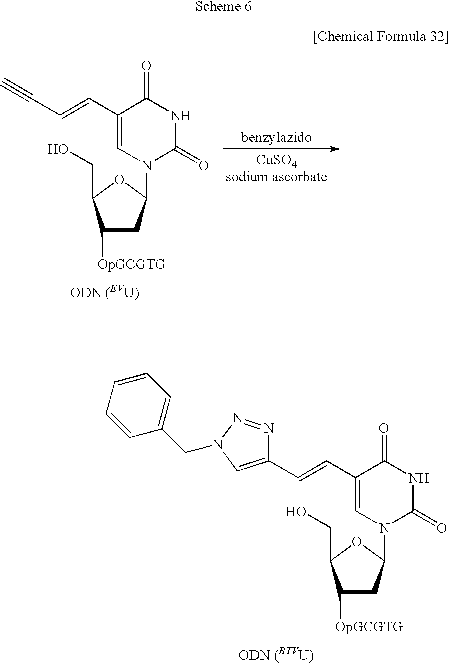

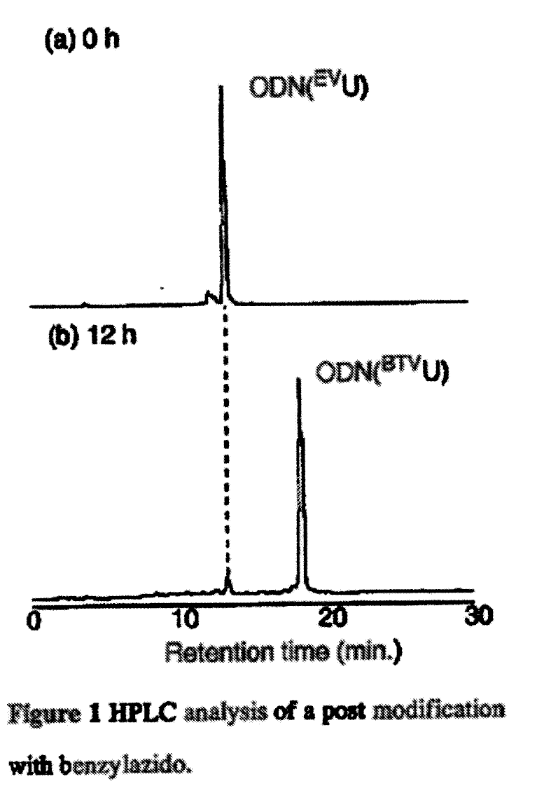

A mixture of a total of 2,000 μl composed of an aqueous solution of ODN (^{EV}U) (50 μM), an aqueous EtOH solution of benzylazido (2.5 mM), an aqueous solution of copper sulfate pentahydrate (2 mM), an aqueous solution of sodium ascorbate (1 mM), and a H₂O: EtOH (=3:1) mixed solution were charged into a screw cap tube, and the post reaction solution obtained 12 hours later was analyzed by HPLC using ammonium formate/acetonitrile (3 to 20%/30 min) (FIG. 1), and the resultant was then fractionated. In this manner, ODN (^{BTV}U) was obtained from ODN including ^{EV}U and benzylazido. The conversion rate was about 90%.

### Click Reaction of ODN Including ^{EV}U with 1-Azidobenzene

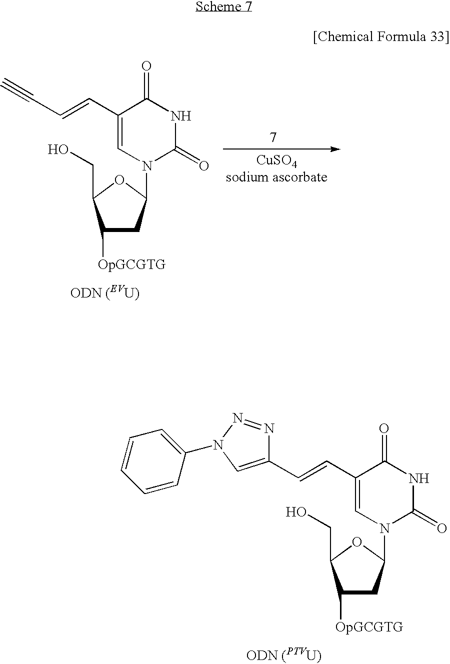

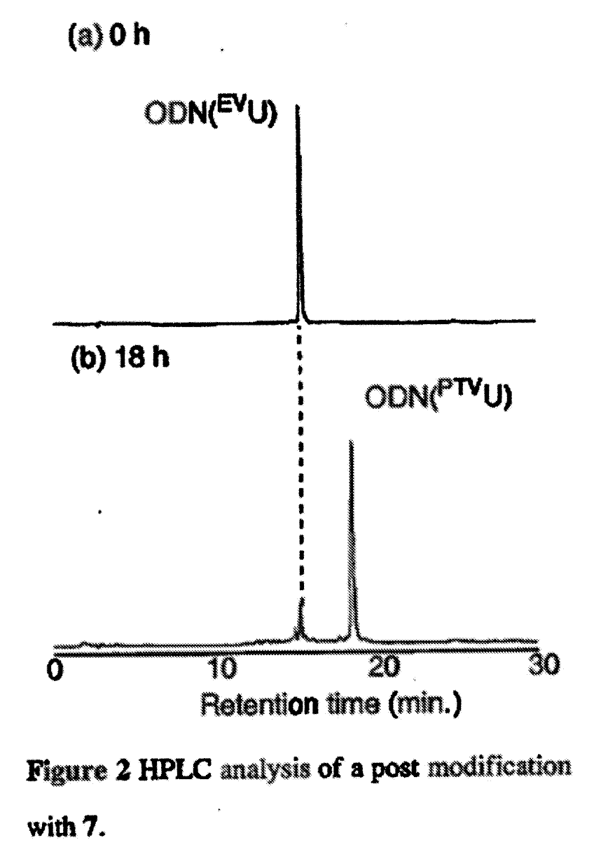

A mixture of a total of 2,000 μl composed of an aqueous solution of ODN (^{EV}U) (50 μM), an aqueous EtOH solution of the compound 7 (2.5 mM), an aqueous solution of copper sulfate pentahydrate (2 mM), an aqueous solution of sodium ascorbate (1 mM), and a H₂O: EtOH (=3:1) mixed solution were charged into a screw cap tube, and the post reaction solution obtained 18 hours later was analyzed by HPLC (FIG. 2). In this manner, ODN (^{PTV}U) was obtained from ODN including ^{EV}U and 1-azidobenzene. The conversion rate was about 90%.

MALDI-TOF MS: Calcd. for ODN (^{PTV}U) [(M+H)⁺] 1978.40, found 1978.42

### Click Reaction of ODN Including ^{EV}U with P-Anisidine

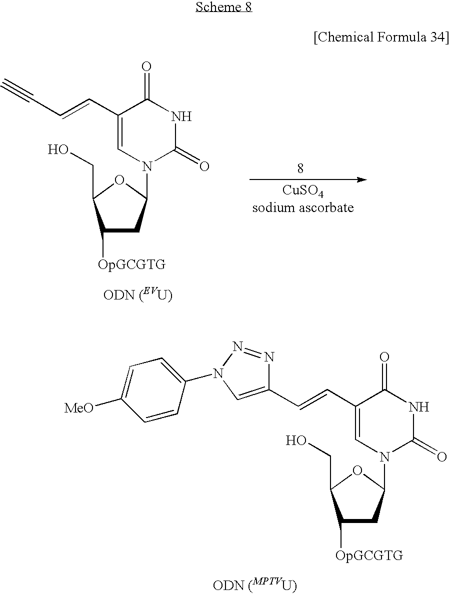

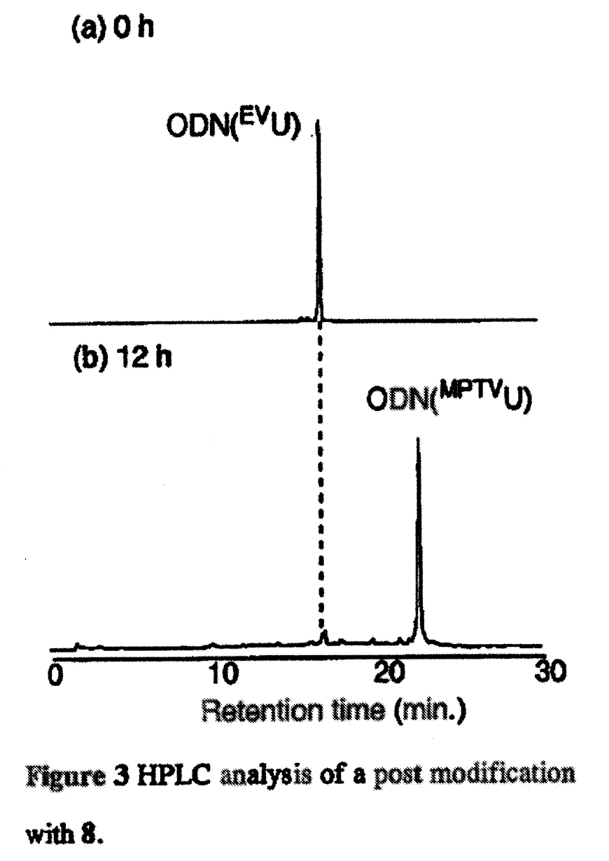

A mixture of a total of 2,000 μl composed of an aqueous solution of ODN (^{EV}U)(50 μM), an aqueous EtOH solution of the compound 8 (2.5 mM), an aqueous solution of copper sulfate pentahydrate (2 mM), an aqueous solution of sodium ascorbate (1 mM), and a H₂O: EtOH (=3:1) mixed solution were charged into a tube, and the post reaction solution obtained 12 hours later was analyzed by HPLC using ammonium formate/acetonitrile (3 to 30%/30 min) (FIG. 3), and the resultant was then fractionated. In this manner, ODN (^{MPTV}U) was obtained from ODN including ^{EV} U and p-anisidine. The conversion rate was about 90%.

MALDI-TOF MS: Calcd. for ODN(^{MPTV}U)[(M−H)⁻] 2006.39, found 2006.38

### Click reaction of ODN Including ^{EV}U with 1-azidobenzonitrile

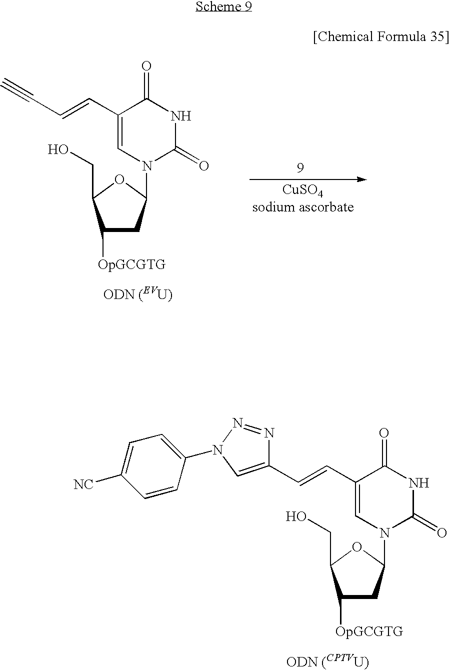

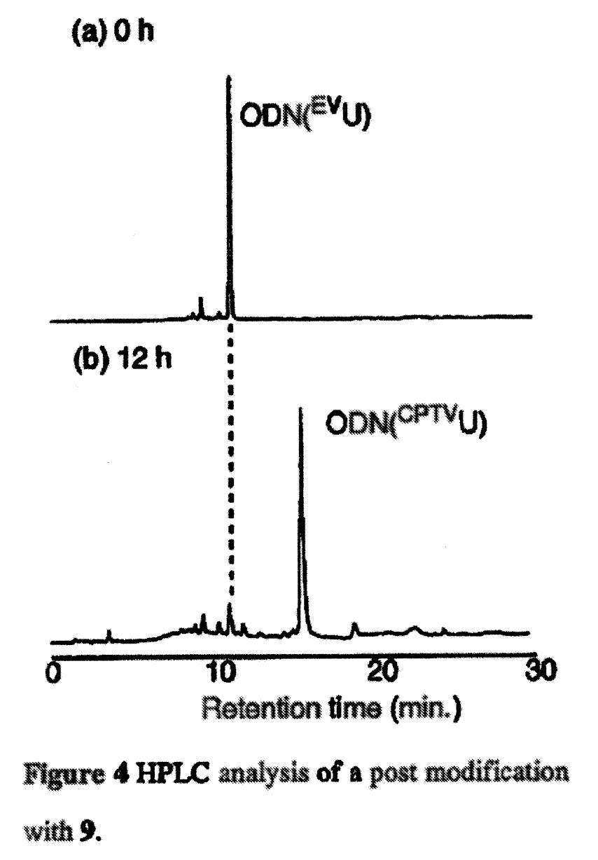

A mixture of a total of 2,000 μl composed of an aqueous solution of ODN (^{EV}U) (50 μM), an aqueous EtOH solution of the compound 9 (2.5 mM), an aqueous solution of copper sulfate pentahydrate (2 mM), an aqueous solution of sodium ascorbate (1 mM), and a H₂O: EtOH (=3:1) mixed solution were charged into a tube, and the post reaction solution obtained 12 hours later was analyzed by HPLC using ammonium formate/acetonitrile (3 to 30%/30 min) (FIG. 4), and the resultant was then fractionated. In this manner, ODN (^{CPTV}U) was obtained from ODN including ^{EV} U and 1-azidobenzonitrile. The conversion rate was about 90%.

### Click Reaction of ODN Including ^{EV}U with 1-naphthylazido

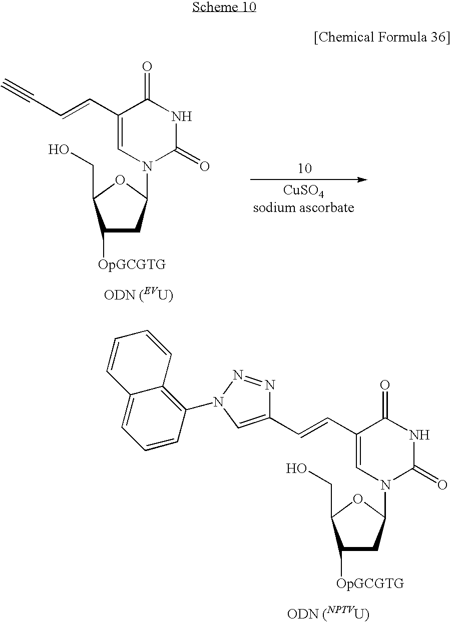

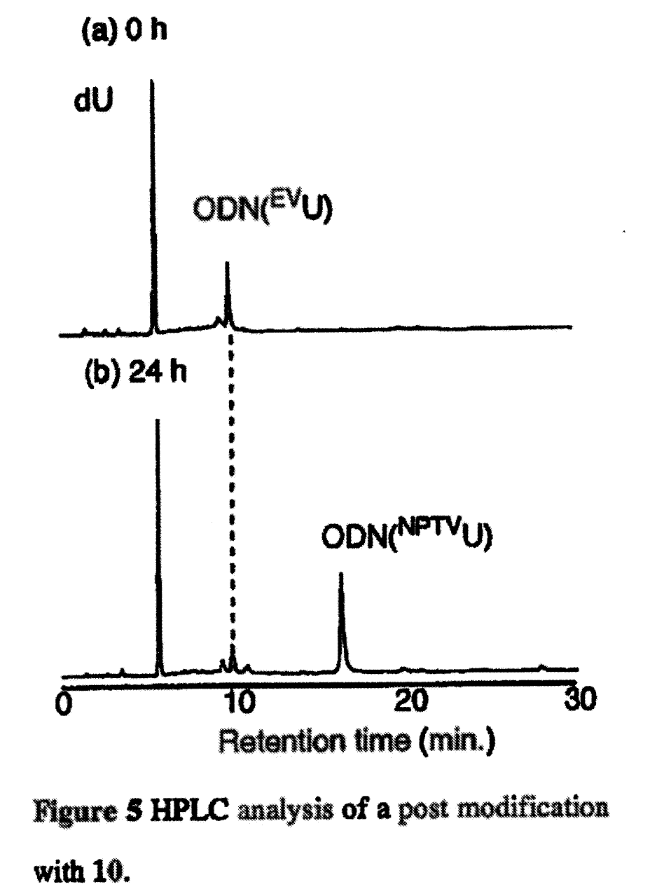

A mixture of a total of 2,000 μl composed of an aqueous solution of ODN (^{EV}U)(50 μM), an aqueous EtOH solution of the compound 10 (2.5 mM), an aqueous solution of copper sulfate pentahydrate (2 mM), an aqueous solution of sodium ascorbate (1 mM), and a H₂O: EtOH (=3:1) mixed solution were charged into a tube, and the post reaction solution obtained 24 hours later was analyzed by HPLC using ammonium formate/acetonitrile (3 to 50%/30 min) (FIG. 5), and the resultant was then fractionated. In this manner, ODN (^{NPTV}U) was obtained from ODN including ^{EV} U and 1-naphthylazido.

[Photocrosslinking Reaction by Photocrosslinkable Nucleic Acids]

[Photocrosslinking reaction using DNA containing ^{BTV}U]

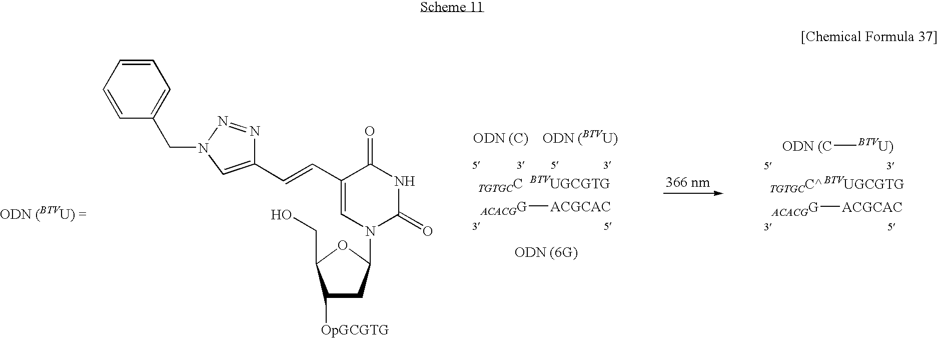

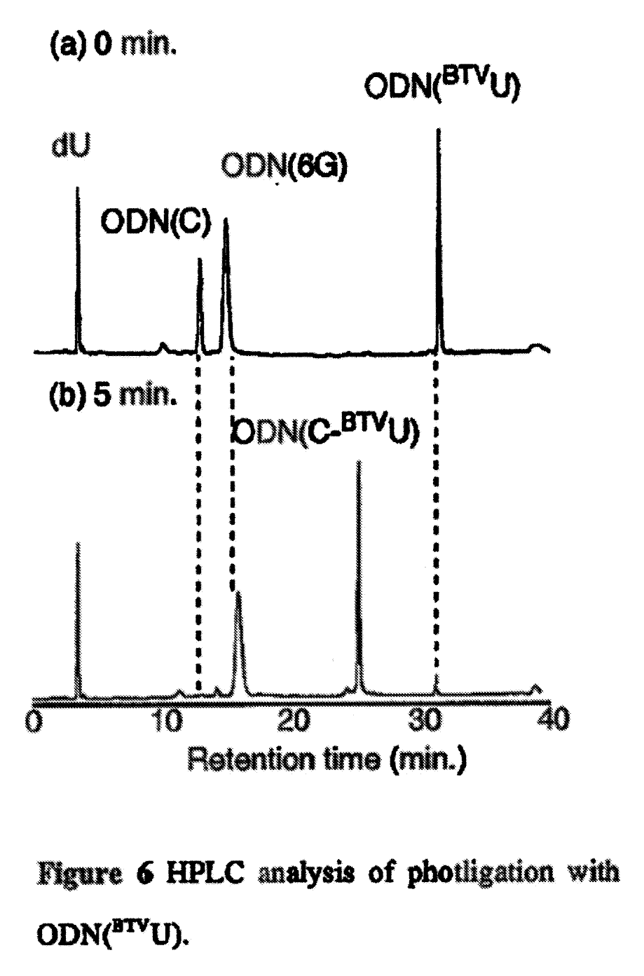

A light irradiation experiment using ODN containing ^{BTV}U was conducted in accordance with Scheme 11. A photocrosslinking reaction of ODN (C) (5′-TGTGCC-3′, 10 μM) with ODN (^{BTV}U) (5′-^{BTV}U GCGTG-3′, 10 μM) was conducted under the presence of 50 mM of sodium cacodylate and 100 mM of NaCl using ODN (6A) (5′-CACGCAGGCACA-3′, 12 μM) as a template nucleic acid (total volume: 200 μl). The reaction product obtained as a result of the photoreaction, in which light having a wavelength of 366 nm was irradiated at 0° C. for 5 minutes using a UV-LED irradiator, was analyzed by HPLC using ammonium formate/acetonitrile (5 to 8%/20 min) (FIG. 6).

[Photocrosslinking Reaction Using DNA Containing ^{PTV}U]

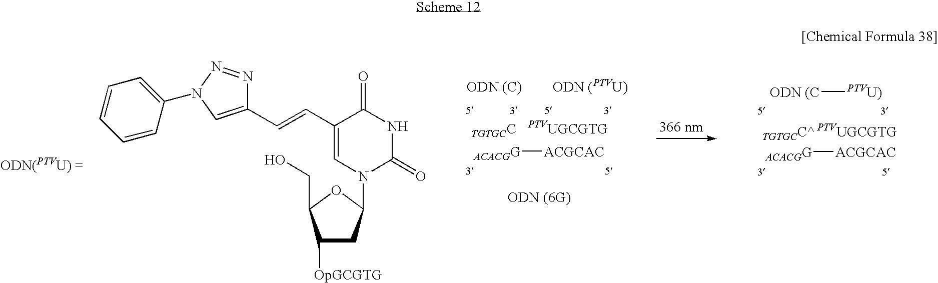

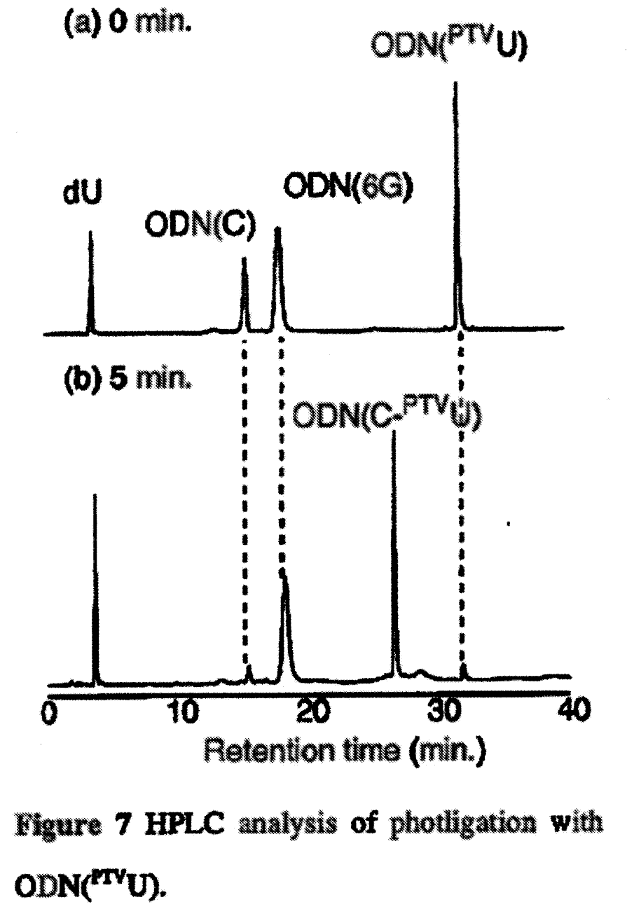

A light irradiation experiment using ODN containing ^{PTV}U was conducted in accordance with Scheme 12. A photocrosslinking reaction of ODN(C) (5′-TGTGCC-3′, 10 μM) with ODN (^{PTV}U) (5′-^{PTV}U GCGTG-3′, 10 μM) was conducted under the presence of 50 mM of sodium cacodylate and 100 mM of NaCl using ODN(6A) (5′-CACGCAGGCACA-3′, 12 μM) as a template nucleic acid (total volume: 200 μl). The reaction product obtained as a result of the photoreaction, in which light having a wavelength of 366 nm was irradiated at 0° C. for 5 minutes using a UV-LED irradiator, was analyzed by HPLC using ammonium formate/acetonitrile (5 to 8%/20 min) (FIG. 7).

[Photocrosslinking Reaction Using DNA Containing ^{MPTV}U]

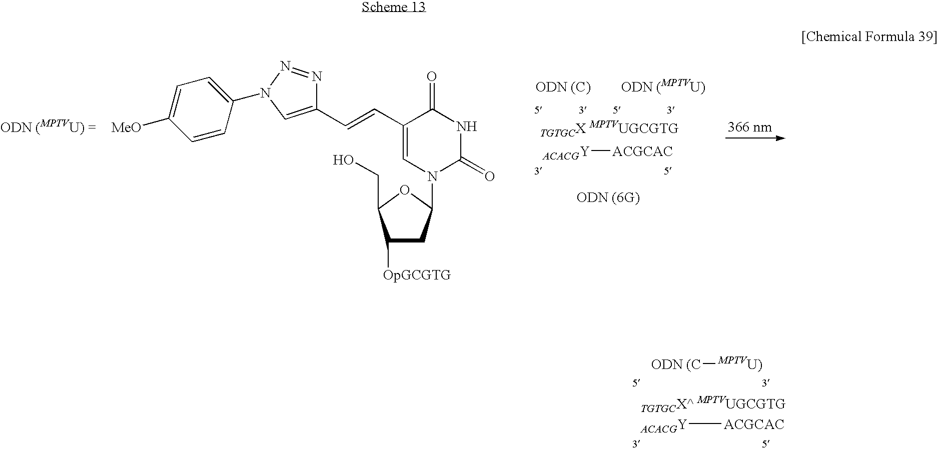

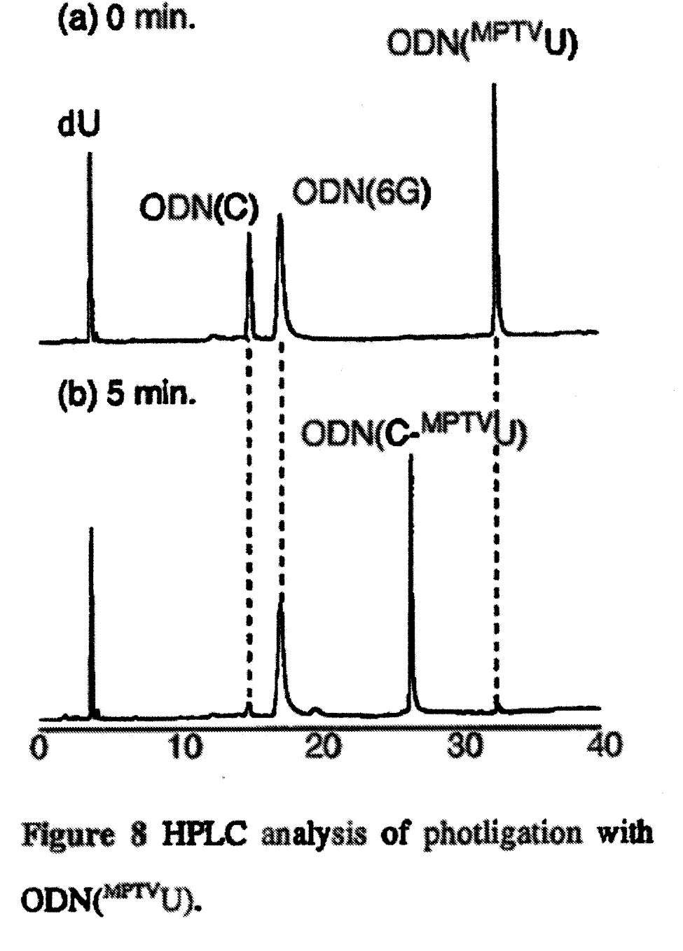

A light irradiation experiment using ODN containing ^{MPTV}U was conducted in accordance with Scheme 13. A photocrosslinking reaction of ODN (C) (5′-TGTGCC-3′, 10 μM) with ODN (^{MPTV}U) (5′-^{MPTV}U GCGTG-3′, 10 μM) was conducted under the presence of 50 mM of sodium cacodylate and 100 mM of NaCl using ODN (6A) (5′-CACGCAGGCACA-3′, 12 μM) as a template nucleic acid (total volume: 200 μl). The reaction product obtained as a result of the photoreaction, in which light having a wavelength of 366 nm was irradiated at 0° C. for 5 minutes using a UV-LED irradiator, was analyzed by HPLC using ammonium formate/acetonitrile (5 to 8%/20 min) (FIG. 8).

[Photocrosslinking Reaction Using DNA Containing ^{CPTV}U]

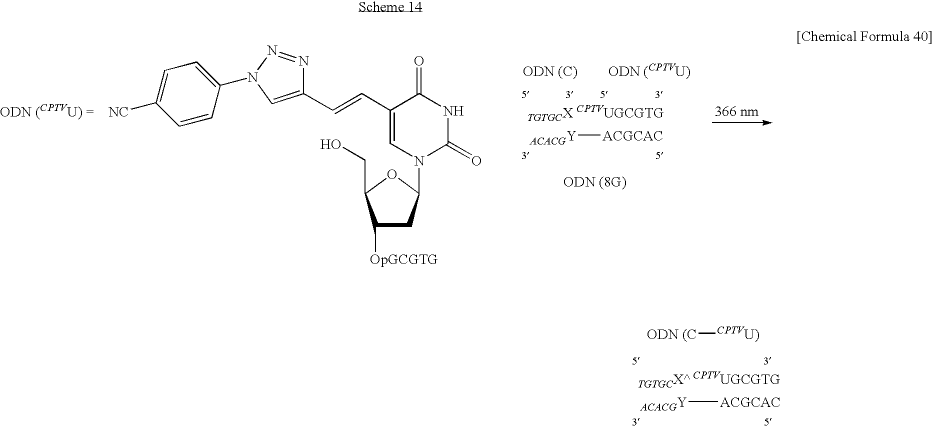

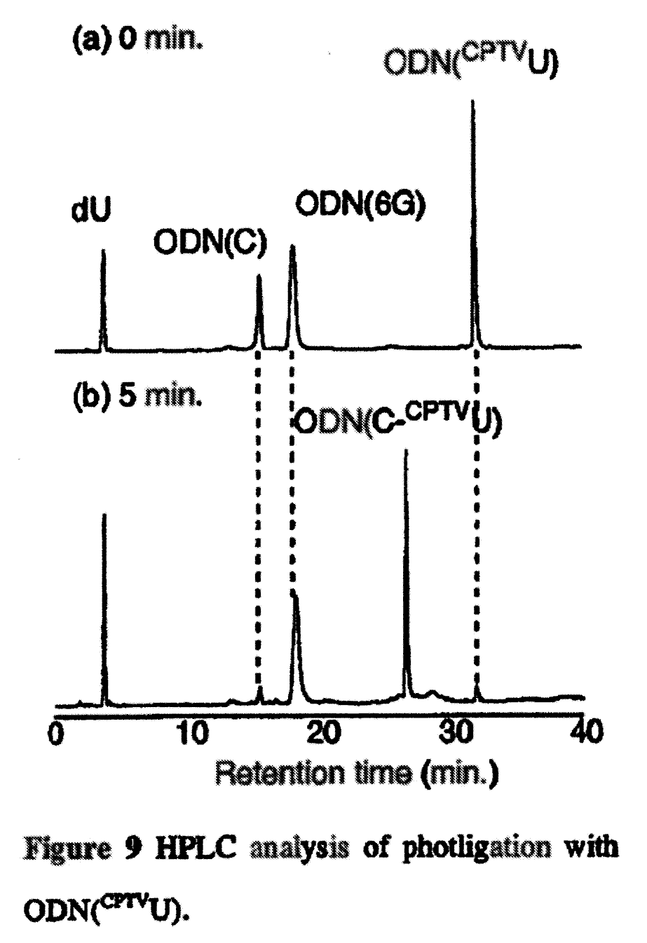

A light irradiation experiment using ODN containing ^{CPTV}U was conducted in accordance with Scheme 14. A photocrosslinking reaction of ODN (C) (5′-TGTGCC-3′, 10 μM) with ODN (^{CPTV}U) (5′-^{CPTV}U GCGTG-3′, 10 μM) was conducted under the presence of 50 mM of sodium cacodylate and 100 mM of NaCl using ODN (6A) (5′-CACGCAGGCACA-3′, 12 μM) as a template nucleic acid (total volume: 200 μl). The reaction product obtained as a result of the photoreaction, in which light having a wavelength of 366 nm was irradiated at 0° C. for 5 minutes using a UV-LED irradiator, was analyzed by HPLC using ammonium formate/acetonitrile (5 to 8%/20 min) (FIG. 9).

[Photocrosslinking reaction using DNA containing ^{NPTV}U]

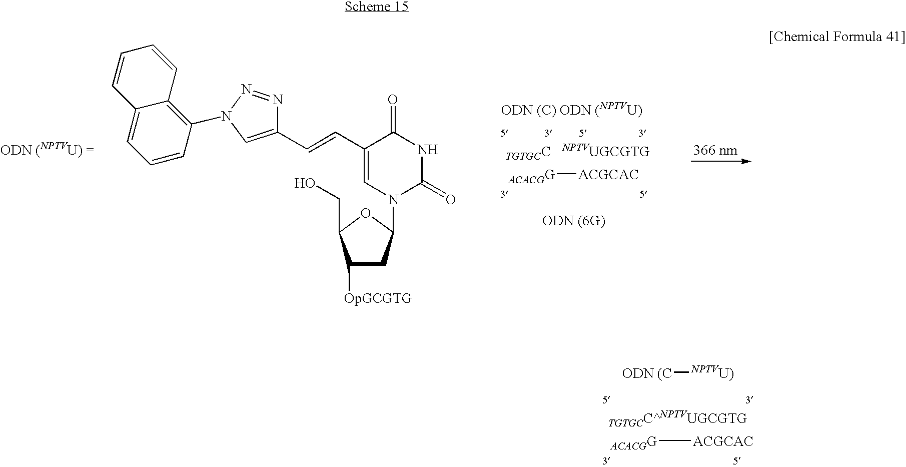

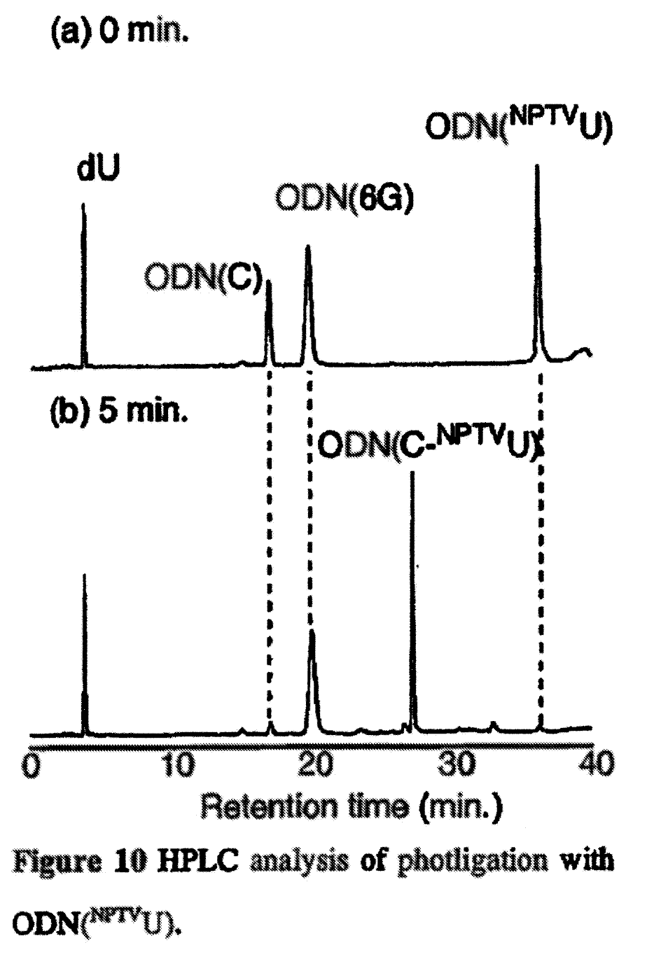

A light irradiation experiment using ODN containing ^{NPTV}U was conducted in accordance with Scheme 15. A photocrosslinking reaction of ODN(C) (5′-TGTGCC-3′, 10 μM) with ODN (^{NPTV}U) (5′-^{NPTV}U GCGTG-3′, 10 μM) was conducted under the presence of 50 mM of sodium cacodylate, 100 mM of NaCl, and 100 μM of a dU monomer using ODN (6A) (5′-CACGCAGGCACA-3′, 12 μM) as a template nucleic acid (total volume: 200 μl). The reaction product obtained as a result of the photoreaction, in which light having a wavelength of 366 nm was irradiated at 0° C. using a UV-LED irradiator, was analyzed by HPLC using ammonium formate/acetonitrile (5 to 8%/20 min, 8 to 50%/20 min) (FIG. 10).

[Photocrosslinking Reaction Using DNA Containing ^{CV}U]

### (Comparative Example)

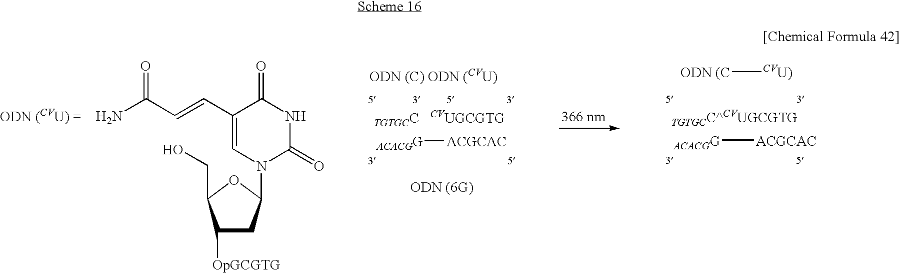

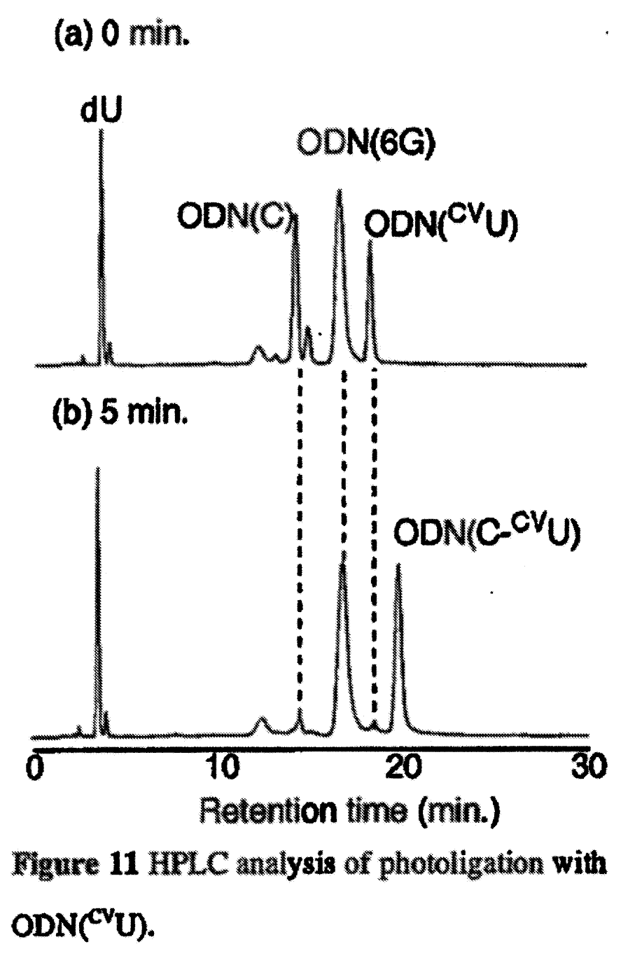

A light irradiation experiment using ODN containing ^{CV}U was conducted in accordance with Scheme 16. A photocrosslinking reaction of ODN(C) (5′-TGTGCC-3′, 10 μM) with ODN (^{CV}U)-^{CV}U GCGTG-3′, 10 μM) was conducted under the presence of 50 mM of sodium cacodylate and 100 mM of NaCl using ODN (6A) (5′-CACGCAGGCACA-3′, 12 μM) as a template nucleic acid (total volume: 200 μl). The reaction product obtained as a result of the photoreaction, in which light having a wavelength of 366 nm was irradiated at 0° C. for 5 minutes using a UV-LED irradiator, was analyzed by HPLC using ammonium formate/acetonitrile (5 to 10%/30 min) (FIG. 11).

[Comparison Result of Photocrosslinking Rate]

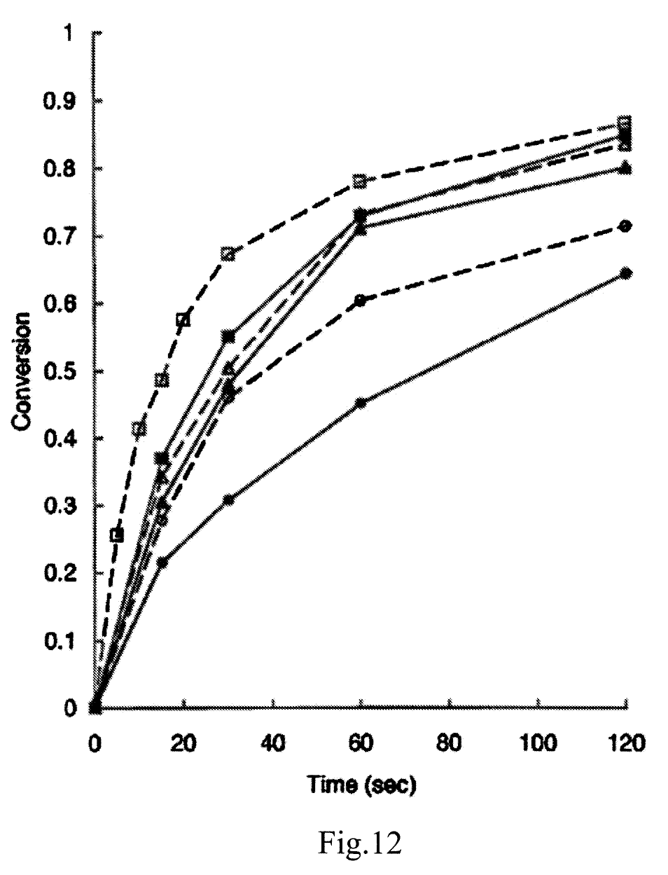

FIG. 12 shows a graph indicating a comparison between the progress of photolinkages of ODN (^{BTV}U) ODN (^{PTV}U) ODN (^{MPTV}U), ODN (^{CPTV}U), and ODN(^{NPTV}U) over time (Example) and the progress of photolinkage of ODN (^{CV}U) over time (Comparative Example). In FIG. 12, the horizontal axis indicates time (seconds) whereas the vertical axis indicate the photolinkage ratio (linking efficiency or conversion efficiency). Data for ODN (^{BTV}U) ODN (^{PTV}U), ODN (^{NPTV}U), ODN (^{CPTV}U), and ODN (^{NPTV}U) were indicated using open circles (O), filled triangles (▴), open triangles (Δ), filled quadrangles (▪), and open quadrangles ( ), respectively.

In FIG. 12, when the times required for achieving a photocrosslinking efficiency of 50% are compared, from the graph curves, those for ODN (^{BTV}U), ODN (^{PTV}U), ODN (^{MPTV}U) and ODN (^{CPTV}U) were about 30 seconds (within a range from 27 to 36 seconds), and that for ODN (^{NPTV}U) was about 17 seconds, whereas that for ODN (^{CV}U) in Comparative Example was about 76 seconds. In other words, compared to ODN (^{CV}U) in Comparative Example, ODN (^{BTV}U), ODN (^{PTV}U), ODN (^{MPTV}U) and ODN (^{CPTV}U) in which 1 benzene ring was conjugated with a triazole structure achieved a photocrosslinking efficiency of 50% in about ½ of time, and ODN (^{NPTV}U) in which 2 benzene rings were conjugated with triazole structures achieved a photocrosslinking efficiency of 50% in about ¼ of time.

Therefore, it became apparent that a photocrosslinking reaction takes place within an extremely short period of time using the nucleic acids according to the present invention, as compared to the conventional photocrosslinkable nucleic acid. In addition, it became clear that the greater the extent of conjugation with the triazole structure (i.e., the greater the number of rings in the aromatic compound), the more dramatically the photocrosslinking rate increases, whereas the presence/absence and type of substituent in the conjugated ring did not affect the photocrosslinking rate as much as the extent of conjugation (i.e., the number of rings in the aromatic compound). In other words, it became clear that when a sufficiently great conjugation system (an aromatic compound) is used, substituents or labeled sites can be provided freely without adversely affecting the progress of photolinkage.

1. Nucleic acids which include a nucleic acid as well as a peptide nucleic acid,
the nucleic acids comprising a group represented by the following formulae I, III, IV, or V as a nucleobase: 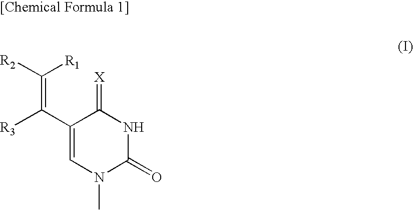
(with the proviso that in the formula I, X represents O, S or NH;
each of R₁ and R₃ independently represents hydrogen, an alkyl group of 1 to 6 carbon atoms, an alkoxy group of 1 to 6 carbon atoms, a cyano group, or an acyl group of 1 to 6 carbon atoms;
and R₂ is a group represented by the following formula II: 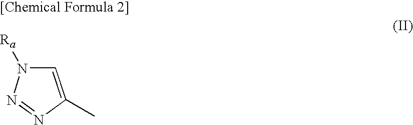
(with the proviso that in the formula II, Ra represents a substituent which can be conjugated with a triazole structure)); 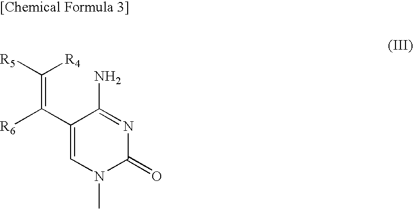
(with the proviso that in the formula III, each of R₄ and R₆ independently represents hydrogen, an alkyl group of 1 to 6 carbon atoms, an alkoxy group of 1 to 6 carbon atoms, a cyano group, or an acyl group of 1 to 6 carbon atoms;
and R₅ is a group represented by the formula II (with the proviso that in the formula II, Ra represents a substituent which can be conjugated with a triazole structure)); 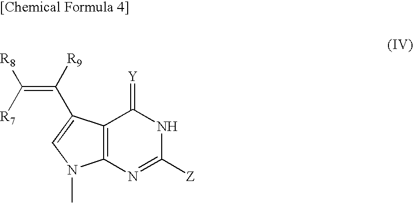
(with the proviso that in the formula IV, Y represents O, S or NH;
Z represents NH₂ when Y represents O or S, and represents a hydrogen atom when Y represents NH;
each of R₇ and R₉ independently represents hydrogen, an alkyl group of 1 to 6 carbon atoms, an alkoxy group of 1 to 6 carbon atoms, a cyano group, or an acyl group of 1 to 6 carbon atoms;
and R₈ is a group represented by the following formula II (with the proviso that in the formula II, Ra represents a substituent which can be conjugated with a triazole structure)); 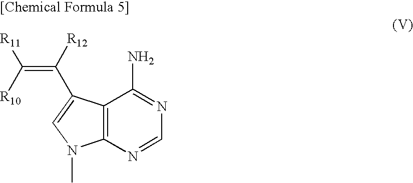
(with the proviso that in the formula V, each of R₁₀ and R₁₂ independently represents hydrogen, an alkyl group of 1 to 6 carbon atoms, an alkoxy group of 1 to 6 carbon atoms, a cyano group, or an acyl group of 1 to 6 carbon atoms;
and R₁₁ is a group represented by the following formula II (with the proviso that in the formula II, Ra represents a substituent which can be conjugated with a triazole structure)).

2. The nucleic acids according to claim 1, wherein Ra is a monovalent group of an aromatic group, having 1, 2, 3 or more rings.

3. A photocrosslinking agent including the nucleic acids according to claim 1.

4. A method for photoligation of nucleic acids using the nucleic acids according to claim 1.

5. A production method of the nucleic acids according to claim 1, the method comprising:
reacting nucleic acids which include a nucleic acid as well as a peptide nucleic acid, with an aromatic azidated product represented by the following formula X: the nucleic acids including a group represented by the following formula VI, formula VII, formula VIII, or formula IX as a nucleobase: 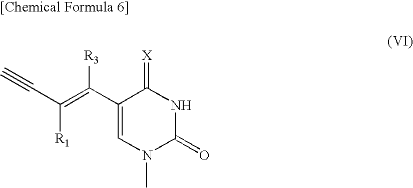
(with the proviso that in the formula VI, X represents O, S or NH;
each of R₁ and R₃ independently represents hydrogen, an alkyl group of 1 to 6 carbon atoms, an alkoxy group of 1 to 6 carbon atoms, a cyano group, or an acyl group of 1 to 6 carbon atoms); 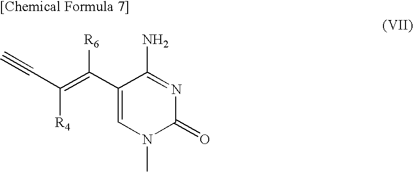
(with the proviso that in the formula VII, each of R₄ and R₆ independently represents hydrogen, an alkyl group of 1 to 6 carbon atoms, an alkoxy group of 1 to 6 carbon atoms, a cyano group, or an acyl group of 1 to 6 carbon atoms); 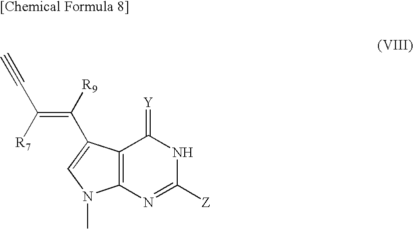
(with the proviso that in the formula VIII, Y represents O, S or NH;
Z represents NH₂ when Y represents O or S, and represents a hydrogen atom when Y represents NH; and
each of R₇ and R₉ independently represents hydrogen, an alkyl group of 1 to 6 carbon atoms, an alkoxy group of 1 to 6 carbon atoms, a cyano group, or an acyl group of 1 to 6 carbon atoms); 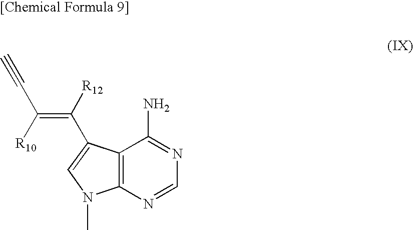
(with the proviso that in the formula IX, each of R₁₀ and R₁₂ independently represents hydrogen, an alkyl group of 1 to 6 carbon atoms, an alkoxy group of 1 to 6 carbon atoms, a cyano group, or an acyl group of 1 to 6 carbon atoms);
with an aromatic azidated product represented by the following formula X:
[Formula 1]
Ra—N₃ (X)
(with the proviso that in the formula X, Ra represents a substituent which can be conjugated with a triazole structure).

6. Nucleic acids which include a nucleic acid as well as a peptide nucleic acid, the nucleic acids comprising a group represented by the following formula VI, formula VII, formula VIII, or formula IX as a nucleobase: 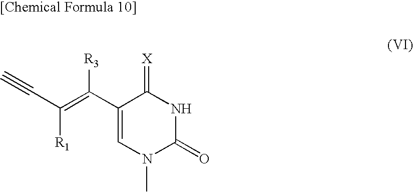
(with the proviso that in the formula VI, X represents O, S or NH; and
each of R₁ and R₃ independently represents hydrogen, an alkyl group of 1 to 6 carbon atoms, an alkoxy group of 1 to 6 carbon atoms, a cyano group, or an acyl group of 1 to 6 carbon atoms); 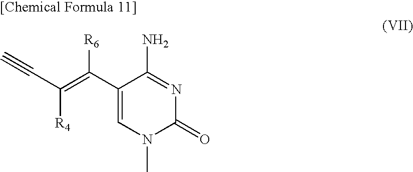
(with the proviso that in the formula VII, each of R₄ and R₆ independently represents hydrogen, an alkyl group of 1 to 6 carbon atoms, an alkoxy group of 1 to 6 carbon atoms, a cyano group, or an acyl group of 1 to 6 carbon atoms); 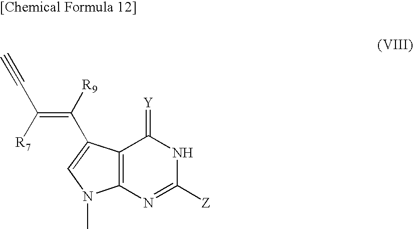
(with the proviso that in the formula VIII, Y represents O, S or NH; and
Z represents NH₂ when Y represents O or S, and represents a hydrogen atom when Y represents NH;
each of R₇ and R₉ independently represents hydrogen, an alkyl group of 1 to 6 carbon atoms, an alkoxy group of 1 to 6 carbon atoms, a cyano group, or an acyl group of 1 to 6 carbon atoms); 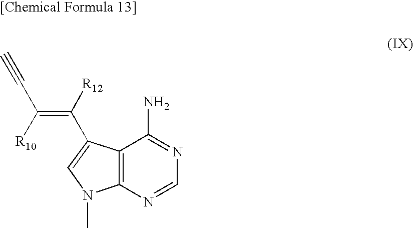
(with the proviso that in the formula IX, each of R₁₀ and R₁₂ independently represents hydrogen, an alkyl group of 1 to 6 carbon atoms, an alkoxy group of 1 to 6 carbon atoms, a cyano group, or an acyl group of 1 to 6 carbon atoms).

7. An aromatic azidated product represented by the following formula X:
[Formula 2]
Ra—N₃ (X)
(with the proviso that in the formula X, Ra represents a substituent which can be conjugated with a triazole structure).

8. A modifying agent for nucleic acids including the aromatic azidated product according to claim 7.

9. A method for modifying nucleic acids using the aromatic azidated product according to claim 7.


SEQUENCE LISTING (9 sequences)

SEQ ID NO: 1 - DNA, 6 bp, Artificial Sequence
  Completely Synthesized
tgtgcc

SEQ ID NO: 2 - DNA, 6 bp, Artificial Sequence
  Completely Synthesized
ugcgtg

SEQ ID NO: 3 - DNA, 12 bp, Artificial Sequence
  Completely Synthesized
cacgcaggcaca

SEQ ID NO: 4 - DNA, 6 bp, Artificial Sequence
  Completely Synthesized
ugcgtg

SEQ ID NO: 5 - DNA, 6 bp, Artificial Sequence
  Completely Synthesized
ugcgtg

SEQ ID NO: 6 - DNA, 6 bp, Artificial Sequence
  Completely Synthesized
ugcgtg

SEQ ID NO: 7 - DNA, 6 bp, Artificial Sequence
  Completely Synthesized
ugcgtg

SEQ ID NO: 8 - DNA, 6 bp, Artificial Sequence
  Completely Synthesized
ugcgtg

SEQ ID NO: 9 - DNA, 6 bp, Artificial Sequence
  Completely Synthesized
ugcgtg